(19) United States
(12) Patent Application Publication
(10) Pub. No.: US 2025/0332239 A1
(43) Pub. Date: Oct. 30, 2025

(54) ANTIGENS FOR CANCER IMMUNOTHERAPY

(71) Applicant: Elixirgen Therapeutics, Inc., Baltimore, MD (US)

(72) Inventor: Minoru S.H. KO, Cockeysville, MD (US)

(73) Assignee: Elixirgen Therapeutics, Inc., Baltimore, MD (US)

(21) Appl. No.: 19/131,488

(22) PCT Filed: Nov. 21, 2023
(86) PCT No.: PCT/US2023/080800

§ 371 Date: May 20, 2025

Related U.S. Application Data
(60) Provisional application No. 63/427,424, filed on Nov. 22, 2022.

(51) Int. Cl. A61K 39/00; A61K 9/00; A61K 47/36; A61P 35/00; C07K 14/705; C12N 9/14; C12N 15/86

(52) U.S. Cl. CPC A61K 39/001164; A61K 9/0019; A61K 47/36; A61P 35/00; C07K 14/70596; C12N 9/14; C12N 15/86; A61K 2039/53; A61K 2039/54; A61K 2039/572; C07K 2319/02; C12N 2770/36134; C12N 2770/36143; C12Y 306/05002

(57) ABSTRACT

The present disclosure relates to expression of fusion proteins for cancer immunotherapy in a mammalian subject, such as a human subject. In particular, the present disclosure relates to mRNA, self-replicating RNA, and temperature-sensitive, self-replicating RNA encoding a plurality of tumor-associated and/or tumor-specific antigens.

### CROSS-REFERENCE TO RELATED APPLICATION(S)

This application claims the benefit of U.S. Provisional Application No. 63/427,424, filed Nov. 22, 2022, which is herein incorporated by reference in its entirety.

### REFERENCE TO AN ELECTRONIC SEQUENCE LISTING

The content of the electronic sequence listing (699442001740SEQLIST.xml; Size: 92,598 bytes; and Date of Creation: Nov. 20, 2023) is herein incorporated by reference in its entirety.

### FIELD

The present disclosure relates to expression of fusion proteins for cancer immunotherapy in a mammalian subject, such as a human subject. In particular, the present disclosure relates to mRNA, self-replicating RNA, and temperature-sensitive, self-replicating RNA encoding a plurality of tumor-associated and/or tumor-specific antigens.

### BACKGROUND

Immunotherapy can be effective in treating cancer and has become more widely used. One therapeutic strategy is to inject immunogenic compositions including antigens that are expressed in tumor cells into cancer patients. Tumor-associated antigens (TAA) are expressed in tumor cells, but are also expressed in embryonic cells or expressed at low levels in normal cells. Tumor-specific antigens (TSA), also called neoantigens, are expressed only in tumor cells, and are often expressed from genes that are mutated in tumor cells. Cancer immunotherapy relies on the induction of a cytotoxic T lymphocyte (CTL) response against cancer cells.

There is a need in the art for cancer immunotherapies that induce potent TAA- or TSA-specific cellular immune responses to destroy tumor cells that express a TAA or a TSA.

### BRIEF SUMMARY

The present disclosure relates to expression of a cancer antigen (TAA and/or TSA) to induce a cellular immune response against cancer cells. In some embodiments, the cancer antigen is encoded by an mRNA. In some embodiments, a temperature-controllable, self-replicating RNA vaccine platform is utilized. In an exemplary embodiment, a cancer antigen is expressed in host cells from a temperature-controllable, self-replicating RNA (c-srRNA) to induce a potent cellular immune response against cancer antigen-expressing tumor cells. A c-srRNA is also referred to herein as a temperature-sensitive self-replicating RNA (srRNAts). The c-srRNA platform described herein is a suitable vector for expression of a tumor-associated antigen (TAA) or a tumor-specific antigen (TSA), also known as a neoantigen. In some embodiments, the TAA is selected from but not limited to the group consisting of NY-ESO-1, MAGEA3, TYR, TPTE (also known as PTEN2) or combinations thereof. In some embodiments, the TSA is an oncoprotein, such as a mutant Ras GTPase. In some embodiments, the mutant Ras GTPase is a KRAS with an activating substitution at one or more of G12, G13, and Q61. c-srRNA is used to express a fusion protein of two or more TAAs, TSAs, or a combination of a TAA and a TSA.

Among other embodiments, the present disclosure provides compositions comprising an excipient and a temperature-controllable, self-replicating RNA (c-srRNA). In some embodiments, the composition comprises a chitosan. In some embodiments, the chitosan is a low molecular weight (about 3-5 kDa) chitosan oligosaccharide, such as chitosan oligosaccharide lactate. In some embodiments, the composition does not comprise liposomes or lipid nanoparticles.

### BRIEF DESCRIPTION OF THE DRAWINGS

FIG. 1 shows a schematic diagram of an exemplary method for designing a fusion protein of mutated 9mer peptide(s). As an example, six common mutations (G12D, G12V, G12R, G12C, G12A, and G12S) of the human KRAS proto-oncogene (GenBank No. NM_001369786) are used. Step 1: Take 9 amino acids (9mer) from a mutated residue (D of the G12D example) in both N-terminal and C-terminal directions to identify a 17 amino acid (17mer) peptide sequence, which includes the mutated residue (in this case D) in the center. If the mutated amino acid is near the end of the N-terminus or C-terminus, one side of the peptide sequence could be shorter than 9mer. Step 2: Repeat the same procedure for other mutations. In this example, six 17mer peptides are generated from six common mutations (G12D, G12V, G12R, G12C, G12A, and G12S) of the human KRAS proto-oncogene. 17mer peptides can be identified from mutations at other locations of the same protein (for example, KRAS) or in other oncoproteins (for example, human TP53 tumor protein p53). Step 3: Generate a fusion protein of the six 17mer peptides. Typically, no additional amino acids are inserted between the 17mer peptide sequences. Alternatively, a non-immunogenic glycine/serine linker can be inserted between one or more of the 17mer peptides. The order of each of the 17mer peptides can be changed from the one shown in this example as SEQ ID NO:29.

FIG. 2 shows a schematic diagram of an exemplary method for designing a fusion protein of mutated 15mer peptide(s). As an example, three common mutations (Q61H, Q61K, and Q61R) of the human KRAS proto-oncogene (GenBank No. NM_001369786) are used. Step 1: Take 15 amino acids (15mer) from a mutated amino acid (H of the Q61H example) in both N-terminal and C-terminal directions to identify a 29 amino acid (29mer) peptide sequence, which includes a mutated amino residue (in this case H) in the center. If the mutated amino acid is near the end of the N-terminus or C-terminus, one side of the peptide sequence could be shorter than 15mer. Step 2: Repeat the same procedure for other mutations. In this example, three 29mer peptides are generated from three common mutations (Q61H, Q61K, and Q61R) of the human KRAS proto-oncogene. 29mer peptides can be obtained from mutations of other locations of the same protein (for example, KRAS) or other proteins (for example, human TP53 tumor protein p53). Step 3: Generate a fusion protein of 29mer peptides. Typically, no additional amino acids are inserted between the 29mer peptide sequences. Alternatively, a non-immunogenic glycine/serine linker can be inserted between one or more of the 29mer peptides. The order of each the 29mer peptides can be changed from the one shown in this example as SEQ ID NO:35.

FIG. 3A shows a schematic diagram of an exemplary fusion protein (TSA-5109) comprising mutated 17mer peptides from human KRAS proto-oncogene (GenBank No. NM_001369786). FIG. 3B shows the amino acid sequences of 13 different 17mer peptides derived from 13 common mutations of human KRAS proto-oncogene (G12D; G12V, G12R, G12C, G12A, G12S, G13D, G13C, G13P, G13S, Q61H, Q61K, and Q61R), which are included in TSA-5109. FIG. 3C shows the amino acid sequence of the TSA-5109 fusion protein including the human CD5 signal peptide (CD5sp) sequence at the N-terminus. The amino acid sequence of the TSA-5109 fusion protein without the CD5sp sequence is set forth as SEQ ID NO: 17, and the amino acid sequence the TSA-5109 fusion protein with the CD5sp sequence is set forth as SEQ ID NO: 18.

FIG. 4A shows a schematic diagram of an exemplary fusion protein (TSA-5111) comprising mutated 29mer peptides from human KRAS proto-oncogene (GenBank No. NM_001369786). FIG. 4B shows the amino acid sequences of 13 different 29mer peptides derived from 13 common mutations of human KRAS proto-oncogene (G12D; G12V, G12R, G12C, G12A, G12S, G13D, G13C, G13P, G13S, Q61H, Q61K, and Q61R), which are included in TSA-5111. FIG. 4C shows the amino acid sequence of the TSA-5111 fusion protein including the human CD5 signal peptide (CD5sp) sequence at the N-terminus. The amino acid sequence of the TSA-5111 fusion protein without the CDSsp sequence is set forth as SEQ ID NO:19, and the amino acid sequence of the TSA-5111 fusion protein with the CD5sp sequence is set forth as SEQ ID NO:20.

FIG. 5 shows a schematic diagram of a fusion protein comprising multiple tumor-associated antigens. In some embodiments, the tumor-associated antigens are expressed from a temperature-controllable self-replicating RNA (c-srRNA). In an exemplary embodiment, the TAA-5107 antigen is a fusion protein comprising the signal peptide sequence from the human CD5 antigen (CDS-SP) set forth as SEQ NO: 1; the amino acid sequence of the human NY-ESO-1 protein set forth as SEQ NO: 4 (GenBank No. NM_001327); the amino acid sequence of the human MAGEA3 protein set forth as SEQ NO: 5 (GenBank No. NM_005362); the amino acid sequence of the human TYR protein (GenBank No. NM_000372); and the amino acid sequence of the human transmembrane phosphatase with tensin homology (TPTE) protein (GenBank No. NM_199261). The amino acid sequence of the TAA-5107 fusion protein (without CD5 SP) is set forth as SEQ ID NO:15, and the amino acid sequence of the CD5-SP plus the TAA fusion protein is set forth as SEQ ID NO:16.

FIG. 6 shows a schematic diagram of an exemplary method for stimulating an immune response against a cancer antigen in a human subject. c-srRNA is functional at a permissive temperature (e.g., 30-35° C.), but non-functional at a non-permissive temperature (e.g., ≥37° C.). The temperature at or just below the surface of a human body (surface body temperature), which is around 31-34° C., is lower than the core body temperature of the human body, which is around 37°° C. The c-srRNA is directly delivered by intradermal and subcutaneous administration to cells of a subject that are at the permissive, surface body temperature.

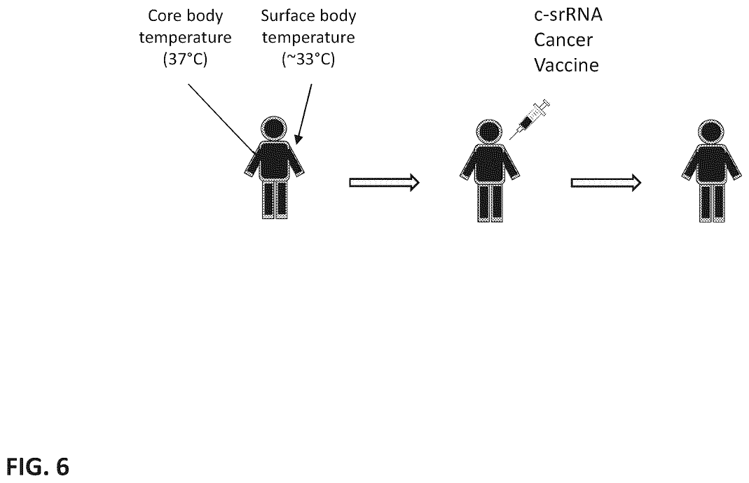

FIG. 7A-7C show the suppression of tumor growth by EXG-5111 vaccine from which the TSA-5111 antigen is expressed in vivo. BALB/c female mice received two intradermal doses of 100 μg of EXG-5111 two-weeks apart. Two weeks later (Day 0), mice received 3×10\hat{ }5 cells of CT26 mouse colon carcinoma cells (ATCC CRL-2638), which are known to have a G12D mutation in KRAS proto-oncogene. FIG. 7A shows the increase in tumor size in 15 mice that received an intradermal placebo (PBO) injection. Mice that met the euthanasia criteria due to tumor size or ulceration were sacrificed. In most mice, tumors rapidly grew and by Day 28 post-tumor injection, only one mouse had survived. FIG. 7B shows the increase in tumor size in 15 mice that received an intradermal EXG-5111 vaccine injection. By contrast to the PBO group, tumor growth was suppressed in mice that received the EXG-5111 vaccine. By Day 28 post-tumor injection, 7 mice had survived. FIG. 7C shows a comparison of tumor growth in PBO and EXG-5111 groups, where the mean±SEM of each group are shown. The suppression of tumor growth by EXG-5111 was statistically significant on Days 11, 14, 22, and 25 as indicated by the asterisk.

### DETAILED DESCRIPTION

Cancer immunotherapy is contemplated to be best achieved through immunogenic compositions that mainly rely on the induction of cellular immunity (i.e., T-cell-inducing vaccines involving CD8+ killer T cells and CD4+ helper T cells). The present disclosure provides mRNA, self-replicating RNA (srRNA), and temperature-controllable, self-replicating RNA (c-srRNA) encoding one or more cancer antigens such as Tumor-associated antigens (TAA) and Tumor-specific antigens (TSA, also called neoantigens). Thus, the present disclosure provides a cellular immunity-based platform for cancer immunotherapy. Wilms tumor 1 (WT1) is a tumor-associated antigen (TAA), which is expressed in a broad range of tumors, but is only expressed in embryonic tissues and very limited cell types in adults. Accordingly, in some embodiments the c-srRNA encodes WT1. In some embodiments, the c-srRNA encodes BIRC5 (aka SURVIVIN). In some embodiments, the c-srRNA encodes NY-ESO-1. In some embodiments, the c-srRNA encodes MAGEA3. In some embodiments, the c-srRNA encodes PRAME. In further embodiments, the c-srRNA encodes one, two, three, four or all five cancer antigens of the group consisting of WT1, BIRC5, NY-ESO-1, MAGEA3, and PRAME.

### Cellular Immunity-Based mRNA Immunotherapeutic Platform

The vaccine platform is described in part in Elixirgen's earlier patent application [PCT/US20/67506, now published as WO 2021/138447 A1]. This vaccine platform is optimized to induce cellular immunity, which becomes possible by combining existing knowledge of vaccine biology with temperature-controllable self-replicating mRNA (c-srRNA) based on an Alphavirus, such as the Venezuelan equine encephalitis virus (VEEV). The terms c-srRNA and srRNAts are used interchangeably throughout the present disclosure, with srRNA1ts2 (described in WO 2021/138447 A1) being an exemplary embodiment. c-srRNA is based on srRNA, which is also known as self-amplifying mRNA (saRNA or SAM), by incorporating small amino acid changes in the Alphavirus replicase that provide temperature-sensitivity. Elixirgen's c-srRNA is functional at a permissive temperature range of about 30-35° C., but is not functional at a non-permissive temperature at or above about 37° C. It carries all the benefits of mRNA platforms: no genome integration, rapid development and deployment, and a simple GMP (good manufacturing process) process, as well as the additional advantages of srRNA platforms (i.e., a predecessor of our c-srRNA platform) compared to mRNA platforms, particularly longer expression [Johanning et al., 1995] and higher immunogenicity at a lower dosage [Brito ct al., 2014]. However, this simple temperature-controllable feature makes it possible to pull together many desirable features of T-cell inducing vaccine as briefly described below.

In brief, srRNA1ts2 is a temperature-sensitive, self-replicating VEEV-based RNA replicon developed for transient expression of a heterologous protein. Temperature-sensitivity is conferred by an insertion of five amino acids residues within the non-structural Protein 2 (nsP2) of VEEV. The nsP2 protein is a helicase/proteinase, which along with nsP1, nsP3 and nsP4 constitutes a VEEV replicase. srRNA1ts2 does not contain VEEV structural proteins (capsid, E1, E2 and E3). The disclosure of WO 2021/138447 A1 of Elixirgen Therapeutics, Inc. is hereby incorporated by reference. In particular, Example 3, FIG. 12, and SEQ ID NOs. 29-49 of WO 2021/138447 A1 are hereby incorporated by reference.

Exemplary vectors include three different temperature-controllable, self-replicating RNA vectors (c-srRNA) and a control self-replicating RNA vector (c-srRNA). Characteristics of the srRNAs suitable for use in the compositions and methods of the present disclosure are summarized in Table I. IFN-α/β sensitivity of the parental VEEV strains was previously reported (Spotts et al., J Viol, 72:10286-10291, 1998). c-srRNA1 was based on the TRD strain of VEEV but modified to have a A16D substitution (TC83 mutation) and a P778S substitution. c-srRNA3 was also based on the TRD strain of VEEV but without the A16D and P778S substitutions. srRNA4 was based on the V198 strain of VEEV, which was isolated from a human. All three c-srRNA vectors include the same 5 amino acid insertion within the nsP2 protein of VEEV for temperature-controllability, as previously described (see U.S. Pat. No. 11,421,248 to Ko, Examples 3, 21 and 22 incorporated herein by reference).

TABLE I

| srRNA Characteristics | | |
|---|---|---|
| RNA | ts-mutant | VEEV |
| srRNA0 | no | TRD |
| c-srRNA1 | yes | TRD/TC-83 |
| c-srRNA3 | yes | TRD |
| c-srRNA4 | yes | V198 |

The nucleotide sequences of the VEEV genomes are disclosed in GenBank: TRD strain as GenBank No. L01442.2; and TC-83 strain as GenBank No. L01443.1. The amino acid sequences of the nsP2 proteins of the srRNAs are disclosed herein: srRNA0 (SEQ ID NO: 13); c-srRNA1 (SEQ ID NO:9); c-srRNA3 (SEQ ID NO:10); c-srRNA4 (SEQ ID NO:11); and c-srRNA consensus (SEQ ID NO:12).

### General Techniques and Definitions

The practice of the present disclosure will employ, unless otherwise indicated, conventional techniques of molecular biology (including recombinant techniques), microbiology, cell biology, biochemistry, and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., molecular weight of about 5,000 daltons when used in reference to a chitosan oligosaccharide refers to 4,500 daltons to 5,500 daltons).

The term “antigen” refers to a substance that is recognized and bound specifically by an antibody or by a T cell antigen receptor. Antigens can include peptides, polypeptides, proteins, glycoproteins, polysaccharides, complex carbohydrates, sugars, gangliosides, lipids, and phospholipids; portions thereof and combinations thereof. In the context of the present disclosure, the term “antigen” typically refers to a polypeptide or protein antigen at least eight amino acid residues in length, which may comprise one or more post-translational modifications.

The terms “polypeptide” and “protein” are used interchangeably to refer to a polymer of amino acid residues, and are not limited to a certain length unless otherwise specified. Polypeptides may include natural amino acid residues or a combination of natural and non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity (e.g., antigenicity).

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that produced the protein. In some embodiments, an isolated protein (e.g., WTI protein) is at least 75%, 90%, 95%, 96%, 97%, 98% or 99% pure as determined by HPLC.

An “effective amount” or a “sufficient amount” of a substance is that amount sufficient to effect beneficial or desired results, including clinical results, and, as such, an “effective amount” depends upon the context in which it is being applied. In the context of administering a composition of the present disclosure comprising an mRNA encoding an antigen, an effective amount contains sufficient mRNA to stimulate an immune response (preferably a cellular immune response against the antigen).

The terms “treating” or “treatment” of a disease refer to executing a protocol, which may include administering one or more drugs to an individual (human or otherwise), in an effort to alleviate a sign or symptom of the disease. Thus, “treating” or “treatment” does not require complete alleviation of signs or symptoms, does not require a cure, and specifically includes protocols that have only a palliative effect on the individual. As used herein, and as well understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission. “Treatment” can also mean prolonging survival of a cancer patient as compared to expected survival of a control patient not receiving treatment. “Palliating” a disease or disorder means that the extent and/or undesirable clinical manifestations of the disease or disorder are lessened and/or time course of progression of the disease or disorder is slowed, as compared to the expected untreated outcome.

In the present disclosure, the terms “individual” and “subject” refer to a mammals. “Mammals” include, but are not limited to, humans, non-human primates (e.g., monkeys), farm animals, sport animals, rodents (e.g., mice and rats) and pets (e.g., dogs and cats). In some preferred embodiments, the subject is a human subject.

The term “dose” as used herein in reference to a composition comprising a mRNA encoding an antigen refers to a measured portion of the taken by (administered to or received by) a subject at any one time. Administering a composition of the present disclosure to a subject in need thereof, comprises administering an effective amount of a composition comprising a mRNA encoding an antigen to stimulate an immune response to the antigen in the subject.

“Stimulation” of a response or parameter includes eliciting and/or enhancing that response or parameter when compared to otherwise same conditions except for a parameter of interest, or alternatively, as compared to another condition (e.g., increase in antigen-specific cytokine secretion after administration of a composition comprising or encoding the antigen as compared to administration of a control composition not comprising or encoding the antigen). For example, “stimulation” of an immune response (e.g., Th1 response) means an increase in the response. Depending upon the parameter measured, the increase may be from 2-fold to 200-fold or over, from 5-fold to 500-fold or over, from 10-fold to 1000-fold or over, or from 2, 5, 10, 50, or 100-fold to 200, 500, 1,000, 5,000, or 10,000-fold.

Conversely, “inhibition” of a response or parameter includes reducing and/or repressing that response or parameter when compared to otherwise same conditions except for a parameter of interest, or alternatively, as compared to another condition. For example, “inhibition” of an immune response (e.g., Th2 response) means a decrease in the response. Depending upon the parameter measured, the decrease may be from 2-fold to 200-fold, from 5-fold to 500-fold or over, from 10-fold to 1000-fold or over, or from 2, 5, 10, 50, or 100-fold to 200, 500, 1,000, 2,000, 5,000, or 10,000-fold.

The relative terms “higher” and “lower” refer to a measurable increase or decrease, respectively, in a response or parameter when compared to otherwise same conditions except for a parameter of interest, or alternatively, as compared to another condition. For instance, a “higher antibody titer” refers to an antigen-reactive antibody titer as a consequence of administration of a composition of the present disclosure comprising an mRNA encoding an antigen that is at least 2, 3, 4, 5, 6, 7, 8, 9, or 10-fold above an antigen-reactive antibody titer as a consequence of a control condition (e.g., administration of a comparator composition that does not comprise the mRNA or comprises a control mRNA that does not encode the antigen). Likewise, a “lower antibody titer” refers to an antigen-reactive antibody titer as a consequence of a control condition (e.g., administration of a comparator composition that does not comprise the mRNA or comprises a control mRNA that does not encode the antigen) that is at least 2, 3, 4, 5, 6, 7, 8, 9, or 10-fold below an antigen-reactive antibody titer as a consequence of administration of a composition of the present disclosure comprising an mRNA encoding an antigen.

As used herein in connection with an agent (e.g., RNA molecule), the term “temperature-sensitive” refers to an agent that has activity at a “permissive temperature”, but has reduced activity at a higher and/or lower “non-permissive temperature.”

As used herein, the term “permissive temperature” refers to any temperature at which the activity of a temperature-sensitive agent of the present disclosure is induced. Typically, a permissive temperature is not the normal body temperature of a subject. The normal body temperature of a human subject is about 37° C.±0.5° C. Depending on the temperature-sensitive agent, a permissive temperature may be a temperature that is higher or lower than the normal body temperature of a subject. In some aspects, the permissive temperature for the temperature-sensitive agent ranges from 30° C. to 36° C. In some embodiments, the permissive temperature is from about 31° C. to about 35° C., or 32° C. to 34° C. (33° C.±1.0° C.). In some preferred embodiments, the permissive temperature is 33° C.±0.5° C. It follows that in some embodiments, the nonpermissive temperature for the temperature-sensitive self-replicating RNAs of the present disclosure is above 36° C. In some preferred embodiments, the non-permissive temperature is 37° C.±0.5° C.

The term “nonpermissive temperature”, as used herein, refers to any temperature at which an activity of a temperature-sensitive agent of the present disclosure is not induced. A temperature-sensitive agent is not induced when an activity of the temperature-sensitive agent is at least 95% less, at least 90% less, at least 85% less, at least 80% less, at least 75% less, or at least 50% less than the level of activity at the optimal permissive temperature. Typically, a non-permissive temperature is the normal body temperature of a subject. Depending on the temperature-sensitive agent, a non-permissive temperature may also be a temperature that is higher (e.g., 38° C. and above) or lower (e.g., below 30° C.) than the normal body temperature of a subject.

As used herein the term “immunization” refers to a process that increases a mammalian subject's reaction to antigen and therefore improves its ability to resist or overcome infection and/or resist disease.

The term “vaccination” as used herein refers to the introduction of a vaccine into

a body of a mammalian subject.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antigen) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

Exemplary amino acid sequences are set forth in sequence identifiers throughout the present disclosure. Some of the claimed embodiments are described by reference to a percent identity shared with an exemplary amino acid sequence. Two amino acid sequences are substantially identical if their amino acid sequences share at least 90% identity (e.g., at least 90%, 91%, 92%, 93%, 94%, 95%, 96%, 97%, 98%, 99% or 100% identity over a specified region, or, when not specified, over their entire sequences), when compared and aligned for maximum correspondence over a comparison window or designated region. As pertains to the present disclosure and claims, the BLASTP sequence comparison algorithm using default parameters is used to align amino acid sequences for determination of sequence identity.

Algorithms that are suitable for determining percent sequence identity and sequence similarity are the BLAST and BLAST 2.0 algorithms, described in Altschul et al., J Mol Biol, 215:403-410, 1990; and Altschul et al., Nucleic Acids Res. 25:3389-3402, 1977, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when; the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff and Henikoff, Proc. Natl. Acad. Sci. USA 89:10915, 1989).

An amino acid substitution may include replacement of one amino acid in a polypeptide with another amino acid. Amino acid substitutions may be introduced into an antigen of interest and the products screened for a desired activity, e.g., increased stability and/or immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

Conservative amino acid substitutions will involve exchanging a member of one of these classes with another member of the same class. Non-conservative amino acid substitutions will involve exchanging a member of one of these classes with a member of another class.

As used herein, the term “excipient” refers to a compound present in a composition comprising an active ingredient (e.g., mRNA encoding an antigen). Pharmaceutically acceptable excipients are inert pharmaceutical compounds, and may include for instance, solvents, bulking agents, buffering agents, tonicity adjusting agents, and preservatives (Pramanick et al., Pharma Times, 45; 65-77, 2013). In some embodiments the compositions of the present disclosure comprise an excipient that functions as one or more of a solvent, a bulking agent, a buffering agent, and a tonicity adjusting agent (e.g., sodium chloride in saline may serve as both an aqueous vehicle and a tonicity adjusting agent).

### Optimized for Intradermal Delivery for Cellular Immunity

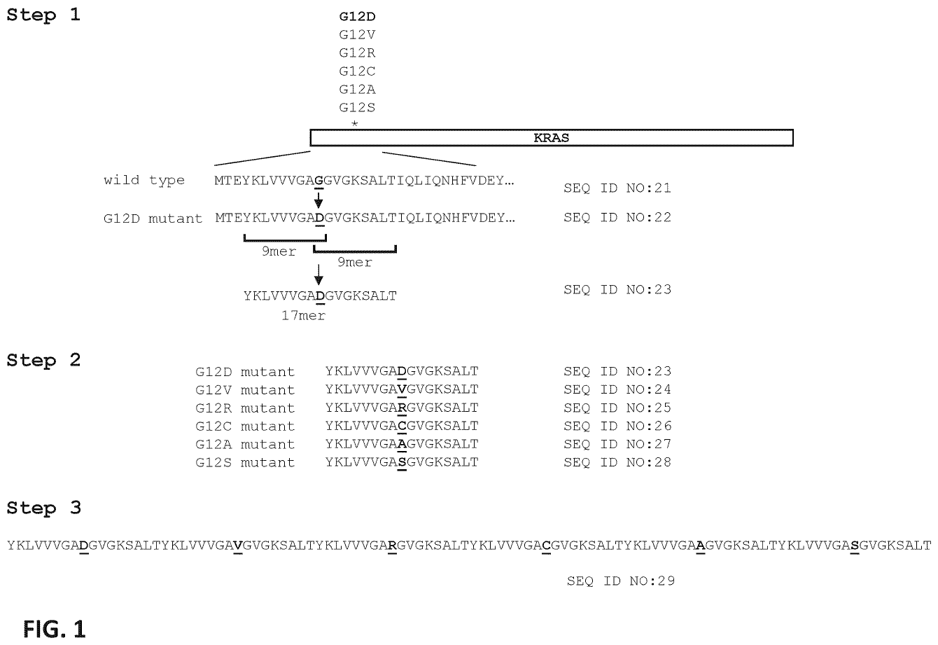

Intradermal vaccination results in long-lasting cellular immunity and increased immunogenicity [Hickling and Jones, 2009]. Human skin (epidermis and dermis) is rich in antigen-presenting cells (APCs), including Langerhans cells and dermal dendritic cells (DCs). Intradermal vaccination is known to be 5- to 10-times more effective than subcutaneous or intramuscular vaccination because it targets the APCs [Hickling and Jones, 2009], and such targeting also activates the T cell immunity pathway for long-lasting immunity. By intradermal injection, c-srRNA is predominantly taken up by skin APCs, wherein it replicates, produces antigen, digests the antigen into peptides, and presents these peptides to T cells (FIG. 1). The peptides presented through this pathway stimulates MHC-I-restricted CD8+ killer T cells. In an alternative pathway, APCs also take antigens produced by nearby skin cells. The peptides presented through this pathway stimulate MHC-II-restricted CD4+ Helper T cells.

### Issues for Intradermal Injection and Solutions

Here are potential issues that we have identified and the solutions that our c-srRNA platform offers.
(1) A key unrecognized hurdle for the application of srRNA as an intradermal vaccine platform is that both mRNA and srRNA do not express antigen well at skin temperature [PCT/US20/67506]. Unintuitively, the temperature of the human skin is lower (about 30-35° C.) than human core body temperature (about 37° C.); this means that vectors and platforms developed at 37° C. are not optimal for intradermal injection. One innovation of our c-srRNA platform is that it expresses antigen strongly at skin temperature [PCT/US20/67506]. Furthermore, this temperature-control also minimizes the safety risk caused by unintended systemic distribution of c-srRNA because c-srRNA becomes inactivated once its temperature increases above its permissive threshold (when it moves closer to the core of the body). In other words, the c-srRNA platform expresses antigen the best for intradermal injection compared to mRNA and srRNA, and it additionally has safety features: the vector's ability to spread and become produced in other areas of a subject's body is limited or inactivated.
(2) Another challenge for intradermal vaccination is the lack of suitable additives. Because adjuvants such as aluminum-salt and oil-in-water are too reactogenic locally when delivered by the intradermal route, no adjuvant has been incorporated into clinically approved intradermal vaccines, resulting in lower immunogenicity [Hickling and Jones, 2009]. Lipid Nanoparticles (LNPs) used for mRNA and srRNA vaccines, which are administered intramuscularly, are also oil-in-water, which may cause skin reactogenicity and increase risk of allergic reactions to LNP components such as PEG. Our c-srRNA platform is a solution to this problem since it is injected as naked c-srRNA (no LNPs, no adjuvants). First, self-replication of RNAs inside cells, especially APCs, induces the strong innate immunity, which substitutes the major functions of adjuvants. Second, data in the literature and of our own demonstrates that, specifically for intradermal injection, naked mRNA/srRNA is equally efficient to produce an antigen compared to electroporation of mRNA/srRNA [Johansson et al, 2012] and mRNA/srRNAs combined with LNPs [Golombek et al., 2018].
(3) A third challenge is the limited number of precedents for intradermal vaccines. Only the BCG vaccine has been administered intradermally on a routine basis. One way we lower the hurdle for adopting intradermal injection is by using specialized devices such as the MicronJet600 (NanoPass) and Immucise (Terumo), which are now available to enable easy, consistent intradermal injection. These devices are also good candidates for large-scale production and deployment. However, due to a relatively high cost of these special devices, an intradermal injection by the Mantoux technique using a standard needle and syringe is also an option.

### Design of Suitable Antigens

A tumor-associated antigens (TAA) is expressed in tumor cells, but also expressed in embryonic cells or expressed at a low level in normal cells. The National Cancer Institute selected 75 cancer antigens that are suitable for a target of cancer therapy (Cheever et al., 2009). For example, Wilms tumor 1 (WT1) ranked as the most promising among the 75 cancer antigens identified by the National Cancer Institute (Cheever et al., 2009). WT1 is expressed in a broad range of tumors, but expressed only in embryonic tissues and very limited cell types in adults. For example, WT1 is expressed in most leukemia (AML, ALL), pancreatic cancer, lung carcinomas, and glioblastoma. Other TAAs can be used as antigen(s) for cancer vaccines based on the c-srRNA platform described herein. It is also possible to use a plurality of TAAs expressed as a fusion protein (Example 3) or a plurality of TAAs expressed separately.

Recently, it has become common to perform genome sequencing of tumor cells derived from patients. Such efforts often identify protein products or peptides that are unique to tumors due to the mutations in their genomes. These Tumor-specific antigens (TSA), also called neoantigens, are ideal targets for cancer vaccine. A single TSA or a fusion of more than one TSA can be used as an antigen for cancer vaccines based on the c-srRNA platform described herein (Examples 1 and 2).

Most tumor-specific antigens (TSA) comprise specific mutation(s), often a single amino acid change in comparison to the normal protein. For example, Glycine (G) to Aspartic acid (D) change at position 12 (G12D) of KRAS is commonly found in human cancers. A challenge here is how to design an antigen that elicits strong T cell immunity, especially CD8+ cytotoxic lymphocytes, against this specific mutation, but not the wild type (normal protein). For the activation of CD8+ cytotoxic lymphocytes, dendritic cells need to present MHC class I molecule loaded with short peptides (typically 9mer, i.e., 9 amino acids). Design of a 17mer peptide, which includes a mutated amino acid in the center is illustrated in FIG. 1, Step 1. In this way, any 9mer peptides processed from the 17mer peptide contain the mutated amino acid, and thus, these 9mer peptides are specific to the mutant protein (neoantigen). If the mutated amino acid is near the end of N-terminus or C-terminus, one side of sequence could be shorter than 9mer. This process is repeated for other mutations to identify a plurality of 17mer sequences comprising the mutations of interest as shown in FIG. 1, Step 2. The 17mer peptide sequences can be identified from other mutations at the same location of the same oncoprotein (e.g., G12D, G12V, and G12R of the KRAS protein), mutations at other locations of the same oncoprotein (e.g., Q61H, Q61K of the KRAS protein), and/or mutations in other oncoproteins (e.g., R175H, R248Q, and R273H of the human TP53 tumor protein p53). Finally, the 17mer peptides are concatenated or expressed as a recombinant fusion protein as shown in FIG. 1, Step 3. Typically, there are no additional amino acids inserted between 17mer peptide sequences. Alternatively, a non-immunogenic glycine/serine linker can be inserted between one or more of the 17mer peptide sequences. The order of each of the 17mer peptide sequences can be changed from the exemplary fusion protein described in Example 1. Thus, exemplary embodiments comprise mRNA molecules encoding KRAS polyproteins in which each neoantigen peptide of the polyprotein is 17 amino acids in length. However, in further embodiments, the mRNA molecules can encode KRAS polyproteins in which each neoantigen peptide of the polyprotein is from 16 to 24 amino acids in length (16, 17, 18, 19, 20, 21, 22, 23 or 24mers).

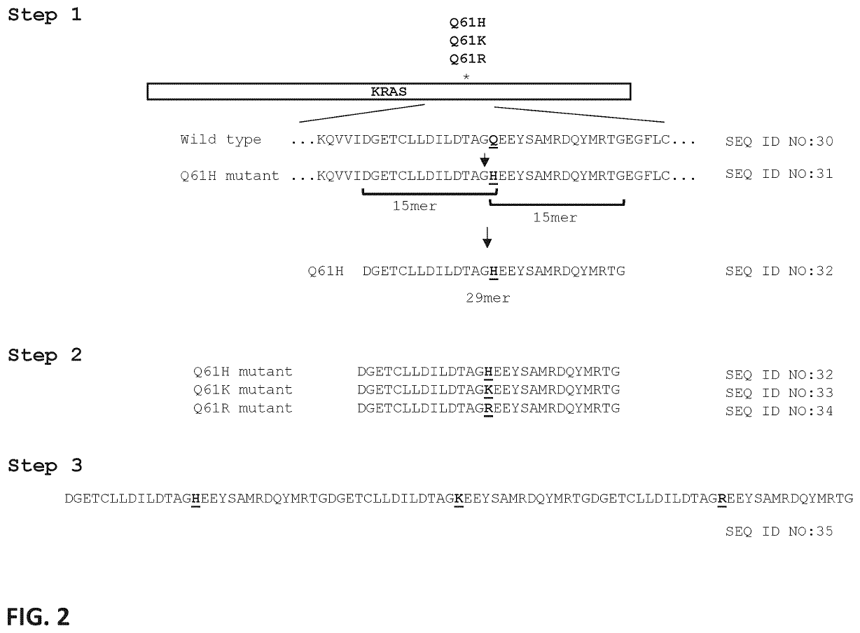

The efficient activation of CD8+ cytotoxic lymphocytes may often require the activation of CD4+ helper T cells. To this end, dendritic cells also need to present MHC class II molecule loaded with longer peptides (typically 15mer, i.e., 15 amino acids). Design of a 29mer peptide, which includes a mutated amino acid in the center is illustrated in FIG. 2, Step 1. In this way, any 15mer peptides processed from the 29mer peptide contain the mutated amino acid, and thus, these 15mer peptides are specific to the mutant protein (neoantigen). Peptides processed from 29mer include 9mer peptides, which are loaded on the MHC class I molecule. Many of these 9mer peptides include the mutated amino acids, but some of the 9mer peptides are wild type. If the mutated amino acid is near the end of N-terminus or C-terminus, one side of sequence could be shorter than 15mer. This process is repeated for other mutations to identify a plurality of 29mer sequences comprising the mutations of interest, as shown in FIG. 2, Step 2. The 29mer peptide sequences can be identified from other mutations of the same location of the same oncoprotein (e.g., G12D, G12V, and G12R of the KRAS protein), mutations at other locations of the same oncoprotein (e.g., Q61H, Q61K of the KRAS protein), and/or mutations in other oncoproteins (e.g., R175H, R248Q, and R273H of the human TP53 tumor protein p53). Finally, the 29mer peptides are concatenated or expressed as a fusion protein as shown in FIG. 2, Step 3. Typically, there are no additional amino acids inserted between 29mer peptide sequences. Alternatively, a non-immunogenic glycine/serine linker can be inserted between one or more of the 29mer peptide sequences. The order of each 29mer peptide sequences can also be changed from the exemplary fusion protein described in Example 2. Thus, exemplary embodiments comprise mRNA molecules encoding KRAS polyproteins in which each neoantigen peptide of the polyprotein is 29 amino acids in length. However, in further embodiments, the mRNA molecules can encode KRAS polyproteins in which each neoantigen peptide of the polyprotein is from 26 to 34 amino acids in length (26, 27, 28, 29, 30, 31, 32, 33 or 34mers).

### Chitosan-Enhancement of Gene Expression in Vivo

An RNase inhibitor (a protein purified from human placenta) slightly enhances the immunogenicity against an antigen encoded on e-srRNA, most likely by enhancing expression of the antigen from the c-srRNA in vivo when intradermally injected into mice (see e.g., FIG. 25C of WO 2021/138447 A1). The RNase inhibitor may protect c-srRNA from RNase-mediated degradation in vivo. However, it is desirable to find an alternative agent that can enhance expression of a gene of interest (GOI) in vivo for therapeutics purposes, as it is difficult to use a protein-based RNase inhibitor as an excipient in injectable products.

A low molecular weight chitosan (molecular weight ˜6 kDa) was shown to inhibit the activity of RNase with the inhibition constants in the range of 30-220 nM (Yakovlev et al., Biochem Biophys Res Commun, 357(3): 584-8, 2007). Two different chitosan oligomers were recently tested: chitosan oligomer (CAS No. 9012-76-4; molecular weight ≤5 kDa, ≥75% deacetylated: Heppe Medical Chitosan GmbH; Product No. 44009), and chitosan oligosaccharide lactate (CAS No. 148411-57-8; molecular weight about 5 kDa, >90% deacetylated: Sigma-Aldrich: Product No. 523682). Surprisingly, even a very low level of chitosan oligomers, as low as 0.001 μg/mL (about 0.2 nM: about 1/100 of the inhibition constant discovered by Yakovlev et al., supra, 2007) was found to be able to enhance the expression of luciferase encoded on c-srRNA by ˜10-fold (data not shown). Similar enhancement of the GOI expression was achieved by chitosan oligomers for up to 0.5 μg/mL and by chitosan oligosaccharide lactate at 0.1 μg/mL.

Chitosan has been used as a nucleotide (DNA and RNA) delivery vector, as it can form complexes or nanoparticles (reviewed in Buschmann et al., Adv Drug Deliv Rev, 65(9):1234-70, 2013; and Cao et al., Drugs, 17:381, 2019). However, it is worth noting that the enhancement of the GOI expression by chitosan oligomers is unlikely to be mediated by the nanoparticle or the complex formation of c-srRNA and chitosan oligomers. First, such a low concentration of chitosan oligomers does not allow the complex formation with RNA. Second, chitosan oligomers are added to c-srRNA immediately before the intradermal injection, and thus, there is not sufficient time to form the complex.

As the chitosan oligomers enhance expression of the GOI in vivo at much lower concentrations compared to the effective concentration as an RNase inhibitor in vitro (Yakovlev et al., supra, 2007), it is conceivable that this enhanced GOI expression by chitosan oligomers may not be mediated by its RNase inhibition mechanism. For example, chitosan oligomers may facilitate the incorporation of c-srRNA into cells, and thereby may enhance the expression of GOI from c-srRNA. Nonetheless, this surprising discovery should provide an effective means to enhance the in vivo therapeutic expression of GOI encoded on c-srRNA.

### ENUMERATED EMBODIMENTS

1. An RNA molecule comprising an open reading frame (ORF) encoding a fusion protein, wherein the ORF comprises from 5′ to 3′;
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:
a) a first segment comprising:

(SEQ ID NO: 53)
MTEYKLVVVGAX₁GVGKSALTIQLIQNXaXbXcXdXeXf
MTEYKLVVVGAX₂GVGKSALTIQLIQNXaXbXcXdXeXf
MTEYKLVVVGAX₃GVGKSALTIQLIQNXaXbXcXdXeXf
MTEYKLVVVGAX₄GVGKSALTIQLIQNXaXbXcXdXeXf
MTEYKLVVVGAX₅GVGKSALTIQLIQNXaXbXcXdXeXf
MTEYKLVVVGAX₆GVGKSALTIQLIQNXaXbXcXdXeXf;

b) a second segment comprising:

(SEQ ID NO: 54)
MTEYKLVVVGAGX₁₀VGKSALTIQLIQNHXaXbXcXdXeXf
MTEYKLVVVGAGX₁₁VGKSALTIQLIQNHXaXbXcXdXeXf
MTEYKLVVVGAGX₁₂VGKSALTIQLIQNHXaXbXcXdXeXf
MTEYKLVVVGAGX₁₃VGKSALTIQLIQNHXaXbXcXdXeXf;

and
c) a third segment comprising:

(SEQ ID NO: 55)
DGETCLLDILDTAGX₇EEYSAMRDQYMRTGXaXbXcXdXeXf
DGETCLLDILDTAGX₈EEYSAMRDQYMRTGXaXbXcXdXeXf
DGETCLLDILDTAGX₉EEYSAMRDQYMRTGXaXbXcXdXeXf,

wherein the first segment, the second segment and the third segment are arranged in any order,
wherein X₁, X₂, X₃, X₄, X₅, and X₆ are independently selected from D, V, R, C, A, and S,
wherein X₇, X₈, and X₉, are independently selected from H, K, and R,
wherein X₁₀, X₁₁, X₁₂, and X₁₃, are independently selected from D, C, P, and S, and
wherein Xa, Xb, Xc, Xd, Xe, and Xf are independently selected from G, S, and absent.
2. The RNA molecule of embodiment 1, wherein the amino acid sequence of the KRAS polyprotein comprises SEQ ID NO:43, SEQ ID NO:44, SEQ ID NO:45, SEQ ID NO:46, SEQ ID NO: 47, SEQ ID NO:48, SEQ ID NO:49, SEQ ID NO:50, SEQ ID NO:51, SEQ ID NO:52, SEQ ID NO:32, SEQ ID NO:33, and SEQ ID NO:34.
3. The RNA molecule of embodiment 2, wherein the amino acid sequence of the KRAS polyprotein comprises residues 25-375 of SEQ ID NO:20.
4. An RNA molecule comprising an open reading frame (ORF) encoding a fusion protein. wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:
a) a first segment comprising:

(SEQ ID NO: 56)
YKLVVVGAX₁GVGKSALTXaXbXcXdXeXfYKLVVVGAX₂GVGKSALTXa
XbXcXdXeXf
YKLVVVGAX₃GVGKSALTXaXbXcXdXeXfYKLVVVGAX₄GVGKSALTXa
XbXcXdXeXf
YKLVVVGAX₅GVGKSALTXaXbXcXdXeXfYKLVVVGAX₆GVGKSALT,

b) a second segment comprising:

(SEQ ID NO: 57)
KLVVVGAGX₁₀VGKSALTIXaXbXcXdXeXfKLVVVGAGX₁₁VGKSALTI
XaXbXcXdXeXf
KLVVVGAGX₁₂VGKSALTIXaXbXcXdXeXfKLVVVGAGX₁₃VGKSALTI,

and
c) a third segment comprising:

(SEQ ID NO: 58)
LDILDTAGX₇HEEYSAMRDXaXbXcXdXeXILDILDTAGX₈HEEYSAMRD
XaXbXcXdXeXf
LDILDTAGX₉HEEYSAMRD,

wherein the first segment, the second segment and the third segment are arranged in any order,
wherein X₁, X₂, X₃, X₄, X₅, and X₆ are independently selected from D, V, R, C, A, and S,
wherein X₇, X₈, and X₉, are independently selected from H, K, and R,
wherein X₁₀, X₁₁, X₁₂, and X₁₃, are independently selected from D, C, P, and S, and
wherein Xa, Xb, Xc, Xd, Xe, and Xf are independently selected from G, S, and absent.
5. The RNA molecule of embodiment 4, wherein the amino acid sequence of the KRAS polyprotein comprises SEQ ID NO:23, SEQ ID NO:24, SEQ ID NO:25, SEQ ID NO:26, SEQ ID NO: 27, SEQ ID NO:28, SEQ ID NO:36, SEQ ID NO:37, SEQ ID NO:38, SEQ ID NO:39, SEQ ID NO:40, SEQ ID NO:41, and SEQ ID NO:42.
6. The RNA molecule of embodiment 5, wherein the amino acid sequence of the KRAS polyprotein comprises residues 25-245 of SEQ ID NO:18.
7. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:

(SEQ ID NO: 56)
YKLVVVGAX₁GVGKSALTXaXbXcXdXeXfYKLVVVGAX₂GVGKSALTXa
XbXcXdXeXf
YKLVVVGAX₃GVGKSALTXaXbXcXdXeXfYKLVVVGAX₄GVGKSALTXa
XbXcXdXeXf
YKLVVVGAX₅GVGKSALTXaXbXcXdXeXfYKLVVVGAX₆GVGKSALT,

wherein X₁, X₂, X₃, X₄, X₅, and X₆ are independently selected from D, V, R, C, A, and S, and
wherein Xa, Xb, Xc, Xd, Xe, and Xf are independently selected from G, S, and absent.
8. The RNA molecule of embodiment 7, wherein the amino acid sequence of the KRAS polyprotein comprises SEQ ID NO:23, SEQ ID NO:24, SEQ ID NO:25, SEQ ID NO:26, SEQ ID NO: 27, and SEQ ID NO:28.
9. The RNA molecule of embodiment 8, wherein the amino acid sequence of the KRAS polyprotein comprises SEQ ID NO:29.
10. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:

(SEQ ID NO: 55)
DGETCLLDILDTAGX₇EEYSAMRDQYMRTGXaXbXcXdXeXf
DGETCLLDILDTAGX₈EEYSAMRDQYMRTGXaXbXcXdXeXf
DGETCLLDILDTAGX₉EEYSAMRDQYMRTGXaXbXcXdXeXf,

wherein X₇, X₈, and X₉, are independently selected from H, K, and R, and
wherein Xa, Xb, Xc, Xd, Xe, and Xf are independently selected from G, S, and absent.
11. The RNA molecule of embodiment 10, wherein the amino acid sequence of the KRAS polyprotein comprises SEQ ID NO:32, SEQ ID NO:33, and SEQ ID NO:34.
12. The RNA molecule of embodiment 11, wherein the amino acid sequence of the KRAS polyprotein comprises SEQ ID NO:35.
13. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:

(SEQ ID NO: 57)
KLVVVGAGX₁₀VGKSALTIXaXbXcXdXeXfKLVVVGAGX₁₁VGKSALTI
XaXbXcXdXeXf
KLVVVGAGX₁₂VGKSALTIXaXbXcXdXeXfKLVVVGAGX₁₃VGKSALT
I,

wherein X₁₀, X₁₁, X₁₂, and X₁₃, are independently selected from D, C, P, and S, and
wherein Xa, Xb, Xc, Xd, Xe, and Xf are independently selected from G, S, and absent.
14. The RNA molecule of embodiment 13, wherein the amino acid sequence of the KRAS polyprotein comprises SEQ ID NO:36, SEQ ID NO:37, SEQ ID NO:38, and SEQ ID NO:39.
15. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:

(SEQ ID NO: 58)
LDILDTAGX₇HEEYSAMRDXaXbXcXdXeXILDILDTAGX₈HEEYSAMRD
XaXbXcXdXeXf
LDILDTAGX₉HEEYSAMRD,

wherein X₇, X₈, and X₉, are independently selected from H, K, and R, and
wherein Xa, Xb, Xc, Xd, Xe, and Xf are independently selected from G, S, and absent.
16. The RNA molecule of embodiment 15, wherein the amino acid sequence of the KRAS polyprotein comprises SEQ ID NO:40, SEQ ID NO:41, and SEQ ID NO:42.
17. The RNA molecule of embodiment 8, embodiment 14, or embodiment 16, wherein the amino acid sequence of the KRAS polyprotein comprises residues 25-245 of SEQ ID NO:18.
18. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the

(SEQ ID NO: 53)
MTEYKLVVVGAX₁GVGKSALTIQLIQNXaXbXcXdXeXf
MTEYKLVVVGAX₂GVGKSALTIQLIQNXaXbXcXdXeXf
MTEYKLVVVGAX₃GVGKSALTIQLIQNXaXbXcXdXeXf
MTEYKLVVVGAX₄GVGKSALTIQLIQNXaXbXcXdXeXf
MTEYKLVVVGAX₅GVGKSALTIQLIQNXaXbXcXdXeXf
MTEYKLVVVGAX₆GVGKSALTIQLIQNXaXbXcXdXeXf,

wherein X₁, X₂, X₃, X₄, X₅, and X₆ are independently selected from D, V, R, C, A, and S, and
wherein Xa, Xb, Xc, Xd, Xe, and Xf are independently selected from G, S, and absent.
19. The RNA molecule of embodiment 18, wherein the amino acid sequence of the KRAS polyprotein comprises SEQ ID NO:43, SEQ ID NO:44, SEQ ID NO:45, SEQ ID NO:46, SEQ ID NO: 47, and SEQ ID NO:48.
20. An RNA molecule comprising an open reading frame (ORF) encoding a fusion protein, wherein the ORF comprises from 5′ to 3″:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:

(SEQ ID NO: 54)
MTEYKLVVVGAGX₁₀VGKSALTIQLIQNHXaXbXcXdXeXf
MTEYKLVVVGAGX₁₁VGKSALTIQLIQNHXaXbXcXdXeXf
MTEYKLVVVGAGX₁₂VGKSALTIQLIQNHXaXbXcXdXeXf
MTEYKLVVVGAGX₁₃VGKSALTIQLIQNHXaXbXcXdXeXf.

wherein X₁₀, X₁₁, X₁₂, and X₁₃, are independently selected from D, C, P, and S, and
wherein Xa, Xb, Xc, Xd, Xe, and Xf are independently selected from G, S, and absent.
21. The RNA molecule of embodiment 20, wherein the amino acid sequence of the KRAS polyprotein comprises SEQ ID NO:49, SEQ ID NO:50, SEQ ID NO:51, and SEQ ID NO:52.
22. The RNA molecule of embodiment 11, embodiment 19, or embodiment 21, wherein the amino acid sequence of the KRAS polyprotein comprises residues 25-375 of SEQ ID NO:20.
23. The RNA molecule of any one of embodiments 1-22, wherein the mammalian signal peptide is a signal peptide of a surface protein expressed in mammalian antigen presenting cells.
24. The RNA molecule of embodiment 23, wherein the mammalian signal peptide is a CD5 signal peptide and the amino acid sequence of the CDS signal peptide comprises SEQ ID NO:1, or the amino acid sequence at least 90% or 95% identical to SEQ ID NO:1.
25. The RNA molecule of any one of embodiments 1-24, comprising at least one modified nucleoside, optionally wherein the at least one modified nucleoside comprises pseudouridine.
26. A DNA template for the RNA molecule of any one of embodiments 1-25, optionally wherein a first restriction enzyme site is present upstream of the nucleotide sequence encoding the mammalian signal peptide, and a second restriction site is present downstream of the nucleotide sequence encoding the cancer antigen.
27. An expression vector comprising the DNA template of embodiment 26.
28. A host cell comprising the expression vector of embodiment 27.
29. The RNA molecule of any one of embodiments 1-25, wherein the RNA molecule is a self-replicating RNA.
30. A composition for stimulating an immune response against a cancer antigen in a mammalian subject, comprising an excipient, and the temperature-sensitive self-replicating RNA of embodiment 29, wherein the self-replicating RNA is a temperature-sensitive RNA that further comprises an Alphavirus replicon lacking a viral structural protein coding region, and wherein the temperature-sensitive self-replicating RNA is capable of expressing the fusion protein at a permissive temperature but not at a non-permissive temperature.
31. A composition for stimulating an immune response against a cancer antigen in a mammalian subject, comprising an excipient, and a temperature-sensitive self-replicating RNA comprising an open reading frame (ORF) encoding a fusion protein, and an Alphavirus replicon lacking a viral structural protein coding region, wherein the ORF comprises from 5′ to 3′:
(i) a nucleotide sequence encoding a mammalian signal peptide; and
(ii) a nucleotide sequence encoding a cancer antigen,
wherein the temperature-sensitive self-replicating RNA is capable of expressing the fusion protein at a permissive temperature but not at a non-permissive temperature, and the cancer antigen comprises a NY-ESO-1 antigen, a MAGEA3 antigen, a TYR antigen, and a TPTE antigen.
32. The composition of embodiment 31, wherein the mammalian signal peptide is a signal peptide of a surface protein expressed in mammalian antigen presenting cells.
33. The composition of embodiment 32, wherein the mammalian signal peptide is a CD5 signal peptide and the amino acid sequence of the CD5 signal peptide comprises SEQ ID NO:1, or the amino acid sequence at least 90% or 95% identical to SEQ ID NO:1.
34. The composition of embodiment 32, wherein the amino acid sequence of the fusion protein comprises SEQ ID NO:16, or the amino acid sequence at least 90% or 95% identical to SEQ ID NO:16.
35. The composition of any one of embodiments 30-34, wherein the Alphavirus is selected from the group consisting of a Venezuelan equine encephalitis virus, a Sindbis virus, and a. Semliki Forrest virus.
36. The composition of embodiment 35, wherein the Alphavirus is a Venezuelan equine encephalitis virus.
37. The composition of any one of embodiments 30-36, wherein the Alphavirus replicon comprises a nonstructural protein coding region with an insertion of 12-18 nucleotides resulting in expression of a nonstructural Protein 2 (nsP2) comprising from 4 to 6 additional amino acids between beta sheet 5 and beta sheet 6 of the nsP2.
38. The composition of embodiment 37, wherein the additional amino acids comprise the sequence of SEQ ID NO: 14 (TGAAA).
39. The composition of embodiment 38, wherein the amino acid sequence of the nsP2 comprises SEQ ID NO:12.
40. The composition of embodiment 39, wherein the amino acid sequence of the nsP2 comprises one sequence selected from the group consisting of SEQ ID NO:9, SEQ ID NO:10, and SEQ ID NO:11.
41. The composition of embodiment 40, wherein the amino acid sequence of the nsP2 comprises SEQ ID NO:11.
42. The composition of any one of embodiments 30-41, wherein the permissive temperature is from 30° C. to 36° C., or 31° C. to 35° C., or 32° C. to 34° C., or 33° C.±0.5° C., and the non-permissive temperature is 37° C.±0.5° C., optionally wherein the permissive temperature is from 31° C. to 35° C. and the non-permissive temperature is at least 37° C.±0.5° C.
43. The composition of any one of embodiments 30-42, wherein the composition does not comprise lipid nanoparticles.
44. The composition of any one of embodiments 30-43, wherein the composition further comprises chitosan.
45. A method for stimulating an immune response against a cancer antigen in a mammalian subject, comprising administering the composition of any one of embodiments 30-44 to a mammalian subject so as to stimulate an immune response against the cancer antigen in the mammalian subject.
46. The method of embodiment 45, wherein the composition is administered intradermally.
47. The method of embodiment 45 or embodiment 46, wherein the immune response comprises a cellular immune response reactive with mammalian cells expressing the cancer antigen.
48. The method of embodiment 47, wherein the cellular immune response comprises one or both of a cancer antigen-specific cytotoxic T lymphocyte response and a cancer antigen-specific helper T lymphocyte response.
49. The method of embodiment 48, wherein the immune response further comprises a humoral immune response reactive with the cancer antigen.
50. The method of any one of embodiments 45-49, wherein the mammalian subject is a human subject.
51. A kit comprising:
(i) the composition of any one of embodiments 30-44; and
(ii) a device for intradermal delivery of the composition to a mammalian subject.
52. The kit of embodiment 51, wherein the device comprises a syringe and a needle.
53. A method of expressing a fusion protein, comprising contacting a mammalian cell with the RNA molecule of any one of embodiments 1-25.
54. The method of embodiment 53, wherein the contacting is in vitro.
55. The method of embodiment 53, wherein the contacting is in vivo.
56. A method of treating cancer, comprising administering an effective amount of the composition of any one of embodiments 30-44 to a mammalian subject in need thereof to treat the cancer.
57. The method of embodiment 56, wherein cells of the cancer express a KRAS oncogene comprising a substitution at one or more of KRAS positions 12, 13 and 61.
58. The method of embodiment 56, wherein cells of the cancer express one or more of a NY-ESO-1 antigen, a MAGEA3 antigen, a TYR antigen, and a TPTE antigen.
59. The method of any one of embodiments 56-58, wherein the composition is administered intradermally.
60. A fusion protein encoded by the RNA molecule of any one of embodiments 1-24, or a mature form of the fusion protein after cleavage of the signal peptide.

### EXAMPLES

Abbreviations: APC (antigen presenting cell); BIRC5 (baculoviral IAP repeat containing 5 or SURVIVIN); GOI (gene of interest); IL-4 (interleukin-4); IFN-γ (interferon gamma); MAGEA3 (melanoma-associated antigen 3); ORF (open reading frame); PBO (placebo); NY-ESO-1 (New York esophageal squamous cell carcinoma 1 or CTAG1B); PRAME (preferentially expressed antigen in melanoma); SFC (spot-forming cells); srRNAts (temperature-sensitive, self-replicating RNA=c-srRNA temperature-controllable, self-replicating RNA); TAA (tumor-associated antigen); TPTE (transmembrane phosphatase with tensin homology); TSA (tumor-specific antigen); TYR (tyrosinase); and WT1 (Wilms tumor 1).

### Example 1. Immunotherapy Against Tumors Expressing KRAS Mutations

This example describes the production of a fusion protein comprising multiple KRAS substitutions based on the design principle illustrated in FIG. 1. A KRAS protein bearing substitutions at one or more of positions 12, 13 and 61 is an exemplary tumor-specific antigen (TSA).

### Materials and Methods

BALB/c inbred female mice.

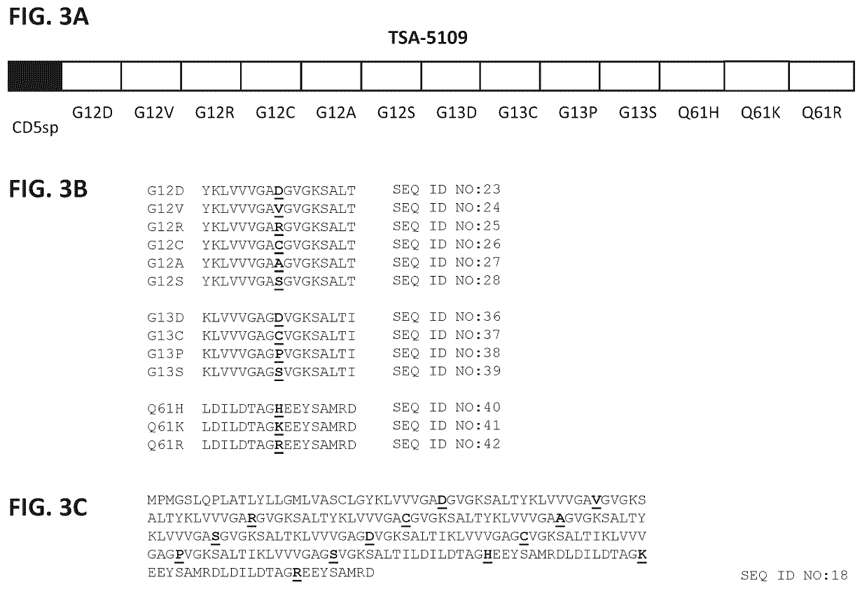

EXG-5109 mRNA was produced by in vitro transcription of a plasmid including a temperature-controllable, self-replicating RNA expression cassette (c-srRNA3) encoding a fusion protein (TSA-5109) comprising 13 different 17mer peptides derived from 13 common mutations of human KRAS proto-oncogene (G12D; G12V, G12R, G12C, G12A, G12S, G13D, G13C, G13P, G13S, Q61H, Q61K, and Q61R). A schematic of the fusion protein is shown in FIG. 3A. The amino acid sequences of the 17mer peptides are shown in FIG. 3B, and the amino acid sequence of the fusion protein including a human CD5 signal peptide is shown in FIG. 3C and set forth in SEQ ID NO:18.

Wild type and mutant peptides shown in Table 1-1 are used to restimulate T cells in splenocyte samples obtained from immunized mice.

TABLE 1-1
Wild Type and Mutant 25mer KRAS Peptides

| | | SEQ |
|---|---|---|
| | | ID |
| Peptide | Sequence | NO: |
| G12G Wild Type | MTEYKLVVVGAGGVGKSALTIQLIQ | 59 |
| G12D Substitution | MTEYKLVVVGADGVGKSALTIQLIQ | 60 |
| G12V Substitution | MTEYKLVVVGAVGVGKSALTIQLIQ | 61 |
| G12C Substitution | MTEYKLVVVGACGVGKSALTIQLIQ | 62 |

The CT26 mouse colon carcinoma cell line (ATCC CRL-2638) is derived from the BALB/c mouse strain and is known to have G12D mutation in KRAS proto-oncogene. The mouse KRAS protein sequence is the same in this region as the human KRAS protein, and thus, the EXG-5109 vaccine developed for humans can be tested in mice. CT26 cells are injected into a BALB/c mouse to form a syngeneic tumor. Either placebo (PBO), 5 μg, or 25 μg of EXG-5109 mRNA vaccine is intradermally administered. Subsequently, tumor sizes are measured.

### Results and Conclusion

The intradermally-injected EXG-5109 mRNA immunotherapeutic is expected to elicit a strong cellular immune response against mutant KRAS proteins, which comprise substitutions at positions 12, 13 and/or 61 of the human KRAS. In ELISpot assays, splenocytes from immunized mice are not expected to respond to the G12G wild type peptide, but are expected to respond to the G12D, G12V, and G12C mutant peptides. In addition, the intradermally-injected EXG-5109 mRNA immunotherapeutic is expected to suppress tumor growth of CT26 mouse colon carcinoma cells in a syngeneic cancer mouse model.

### Example 2. Immunotherapy Against Tumors Expressing KRAS Mutations

This example describes the production of a fusion protein comprising multiple KRAS substitutions based on the design principle illustrated in FIG. 2. A KRAS protein bearing substitutions at one or more of positions 12, 13 and 61 is an exemplary tumor-specific antigen (TSA).

### Materials and Methods

BALB/c inbred female mice.

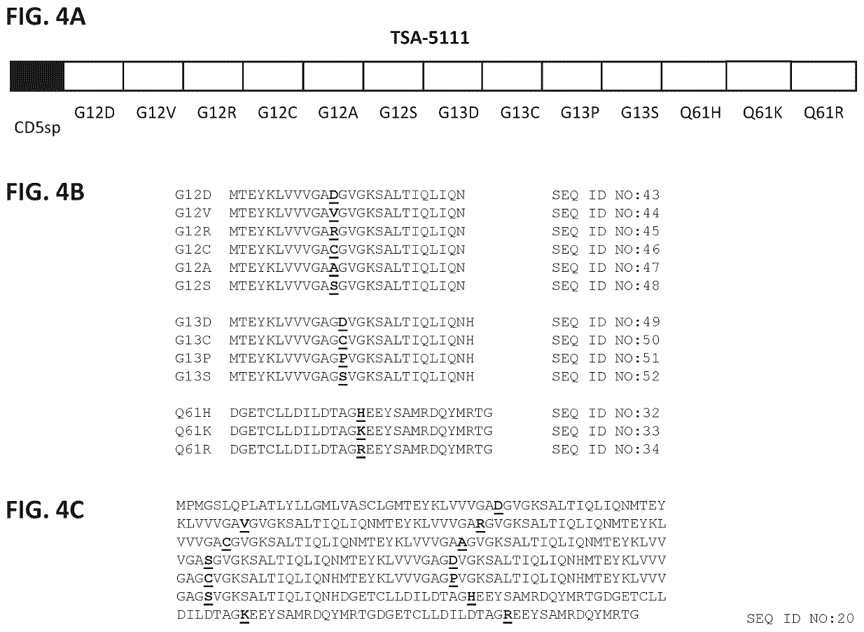

EXG-5111 mRNA was produced by in vitro transcription of a plasmid including a temperature-controllable, self-replicating RNA expression cassette (c-srRNA3) encoding a fusion protein (TSA-5111) comprising 13 different peptides (each from 26-29 amino acids in length) derived from 13 common mutations of human KRAS proto-oncogene (G12D; G12V, G12R, G12C, G12A, G12S, G13D, G13C, G13P, G13S, Q61H, Q61K, and Q61R). A schematic of the fusion protein is shown in FIG. 4A. The amino acid sequences of the 26-29mer peptides are shown in FIG. 4B, and the amino acid sequence of the fusion protein including a human CD5 signal peptide is shown in FIG, 4C and set forth in SEQ ID NO:20.

Wild type and mutant peptides shown in Table 1-1 were used to restimulate T cells in splenocyte samples obtained from immunized mice.

The CT26 mouse colon carcinoma cell line (ATCC CRL-2638) is derived from the BALB/c mouse strain and is known to have G12D mutation in KRAS proto-oncogene. The mouse KRAS protein sequence is the same in this region as the human KRAS protein, and thus, the EXG-5111 vaccine developed for humans can be tested in mice. CT26 cells were injected into a BALB/c mouse to form a syngeneic tumor. Either placebo (PBO), 5 μg, or 25 μg of EXG-5111 mRNA vaccine was intradermally administered. Subsequently, tumor sizes were measured.

### Results and Conclusion

The intradermally-injected EXG-5111 immunotherapeutic is expected to elicit a strong cellular immune response against mutant KRAS proteins, which comprise substitutions at positions 12, 13 and/or 61 of the human KRAS. In ELISpot assays, splenocytes from immunized mice are not expected to respond to the G12G wild type peptide, but are expected to respond to the G12D, G12V, and G12C mutant peptides.

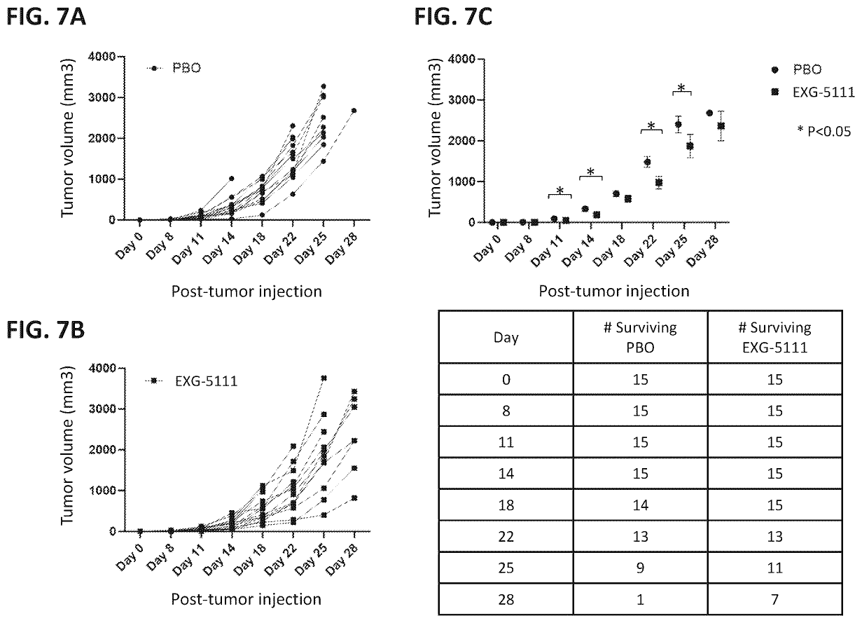

As expected, the intradermally-injected EXG-5111 mRNA immunotherapeutic suppressed tumor growth of CT26 mouse colon carcinoma cells in a syngeneic cancer mouse model. BALB/c female mice received two intradermal doses of 100 μg of EXG-5111 two-weeks apart. Two weeks later (Day 0), mice received 3×10\hat{ }5 cells of CT26 mouse colon carcinoma cells (ATCC CRL-2638), which are known to have G12D mutation in KRAS proto-oncogene. FIG. 7A shows the increase in tumor size (volume) in 15 mice that received intradermal placebo (PBO) injection. Mice that met the euthanasia criteria due to tumor size or ulceration were sacrificed. In general, tumors rapidly grew in placebo-treated mice and by Day 28 post-tumor injection, only one mouse had survived. FIG. 7B shows the increase in tumor size (volume) in 15 mice that received intradermal EXG-5111 vaccine injection. By contrast to the PBO, tumor growth was suppressed and slower in mice that received EXG-5111 vaccine. By Day 28 post-tumor injection, 7 mice had survived. FIG. 7C shows the comparison of PBO and EXG-5111 group, where the mean±SEM of each group are shown in the graph with numbers of surviving mice in each group shown below. The suppression of tumor growth by EXG-5111 was statistically significant on Days 11, 14, 22, and 25.

### Example 3. Immunotherapy Against Tumors Expressing a Plurality of Tumor-Associated Antigens (TAAs)

This example describes assessing whether intradermally-injected c-srRNA encoding a fusion protein (TAA-5107) comprising a human CD5 signal peptide, NY-ESO-1, MAGEA3, TYR and TPTE is able to induce potent cellular immune responses in BALB/c mice against the TAAs of the fusion protein.

### Materials and Methods

BALB/c inbred female mice.

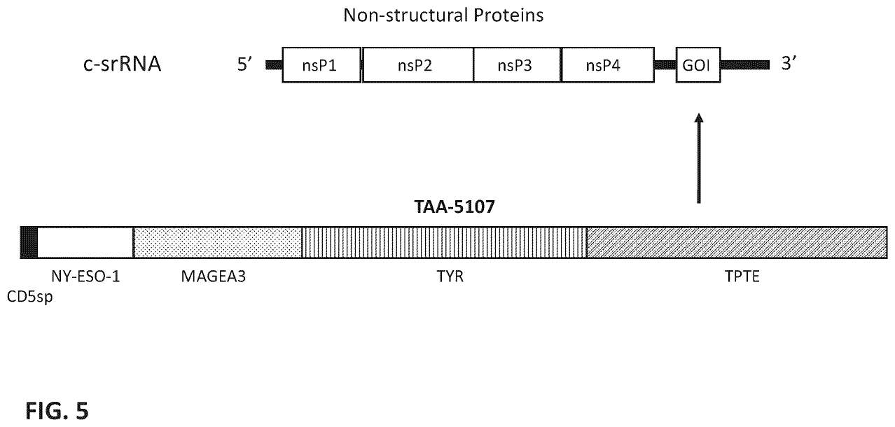

FIG. 5 shows a schematic diagram of the EXG-5107 vaccine. EXG-5107 mRNA was produced by in vitro transcription of a plasmid including a temperature-controllable, self-replicating RNA expression cassette (c-srRNA3) encoding a fusion protein (TAA-5107) comprising the human CD5 signal peptide, NY-ESO-1, MAGEA3, TYR, and TPTE. The amino acid sequences of the fusion protein including a human CD5 signal peptide is set forth as SEQ ID NO: 16.

Either placebo (PBO) or 25 μg of the EXG-5107 vaccine is intradermally administered. Subsequently, cellular immunity against the TAAs of the TAA-5107 fusion protein is assessed by ELISpot assays.

### Results and Conclusion

The intradermally-injected EXG-5107 mRNA immunotherapeutic is contemplated to elicit strong cellular immune responses against distinct components of the fusion protein (NY-ESO-1, MAGEA3, TYR, and TPTE).

### REFERENCES

References pertaining to the present disclosure include: PCT/US2022/075789 and PCT/US2020/067506 of Elixirgen Therapeutics, Inc., the examples of which are incorporated herein by reference. Additional references pertaining to the present disclosure include: Brito et al., Mol Ther. 22(12): 2118-2129, 2014; Cheever et al., Clin Cancer Res. 15:5323-5337, 2009; Golombek et al., Mol Ther Nucleic Acids. 11:382-392, 2018; Hickling et al., Intradermal Delivery of Vaccines: A review of the literature and the potential for development for use in low-and middle-income countries. PATH/WHO Aug. 27, 2009; Johanning et al., Nucleic Acids Res. 23(9): 1495-501, 1995; and Johansson et al., PLOS One. 7(1): e29732, 2012.

SEQUENCES
>Human CD5 Signal Peptide
SEQ ID NO: 1
MPMGSLQPLATLYLLGMLVASCLG
>Human Wilms tumor protein (NM_024426.6)
SEQ ID NO: 2
MDFLLLQDPASTCVPEPASQHTLRSGPGCLQQPEQQGVRDPGGIWAKLGAAEASAERLQGRRSRGASGSE
PQQMGSDVRDLNALLPAVPSLGGGGGCALPVSGAAQWAPVLDFAPPGASAYGSLGGPAPPPAPPPPPPPP
PHSFIKQEPSWGGAEPHEEQCLSAFTVHFSGQFTGTAGACRYGPFGPPPPSQASSGQARMFPNAPYLPSC
LESQPAIRNQGYSTVTFDGTPSYGHTPSHHAAQFPNHSFKHEDPMGQQGSLGEQQYSVPPPVYGCHIPTD
SCTGSQALLLRTPYSSDNLYQMTSQLECMTWNQMNLGATLKGVAAGSSSSVKWTEGQSNHSTGYESDNHT
TPILCGAQYRIHTHGVERGIQDVRRVPGVAPTLVRSASETSEKRPFMCAYPGCNKRYFKLSHLQMHSRKH
TGEKPYQCDFKDCERRFSRSDQLKRHQRRHTGVKPFQCKTCQRKFSRSDELKTHTRTHIGKISEKPFSCR
WPSCQKKFARSDELVRHHNMHQRNMTKLQLAL
>Human BIRC5 (AKA SURVIVIN) protein (NM_001168)
SEQ ID NO: 3
MGAPTLPPAWQPFLKDHRISTFKNWPFLEGCACTPERMAEAGFIHCPTENEPDLAQCFFCFKELEGWEPD
DDPIEEHKKHSSGCAFLSVKKQFEELILGEFLKLDRERAKNKIAKETNNKKKEFEETAEKVRRAIEQLAA
MD
>Human NY-ESO-1 protein (NM_001327)
SEQ ID NO: 4
MQAEGRGIGGSTGDADGPGGPGIPDGPGGNAGGPGEAGATGGRGPRGAGAARASGPGGGAPRGPHGGAAS
GLNGCCRCGARGPESRLLEFYLAMPFATPMEAELARRSLAQDAPPLPVPGVLLKEFTVSGNILTIRLTAA
DHRQLQLSISSCLQQLSLLMWITQCFLPVFLAQPPSGQRR
>Human MAGEA3 protein (NM_005362)
SEQ ID NO: 5
MPLEQRSQHCKPEEGLEARGEALGLVGAQAPATEEQEAASSSSTLVEVTLGEVPAAESPDPPQSPQGASS
LPTTMNYPLWSQSYEDSSNQEEEGPSTFPDLESEFQAALSRKVAELVHFLLLKYRAREPVTKAEMLGSVV
GNWQYFFPVIFSKASSSLQLVFGIELMEVDPIGHLYIFATCLGLSYDGLLGDNQIMPKAGLLIIVLAIIA
REGDCAPEEKIWEELSVLEVFEGREDSILGDPKKLLTQHEVQENYLEYRQVPGSDPACYEFLWGPRALVE
TSYVKVLHHMVKISGGPHISYPPLHEWVLREGEE
>Human PRAME protein (NM_001291715)
SEQ ID NO: 6
MERRRLWGSIQSRYISMSVWTSPRRIVELAGQSLLKDEALAIAALELLPRELFPPLFMAAFDGRHSQTLK
AMVQAWPFTCLPLGVLMKGQHLHLETFKAVLDGLDVLLAQEVRPRRWKLQVLDLRKNSHQDFWTVWSGNR
ASLYSFPEPEAAQPMTKKRKVDGLSTEAEQPFIPVEVLVDLFLKEGACDELFSYLIEKVKRKKNVLRLCC
KKLKIFAMPMQDIKMILKMVQLDSIEDLEVTCTWKLPTLAKFSPYLGQMINLRRLLLSHIHASSYISPEK
EEQYIAQFTSQFLSLQCLQALYVDSLFFLRGRLDQLLRHVMNPLEILSIINCRLSEGDVMHLSQSPSVSQ
LSVLSLSGVMLTDVSPEPLQALLERASATLQDLVFDECGITDDQLLALLPSLSHCSQLITLSFYGNSISI
SALQSLLQHLIGLSNLTHVLYPVPLESYEDIHGTLHLERLAYLHARLRELLCELGRPSMVWLSANPCPHC
GDRTFYDPEPILCPCFMPN
>ARTIFICIAL: FUSION OF WT1, BIRC5, NY-ESO-1, MAGEA3, and PRAME
SEQ ID NO: 7
LDFLLLQDPASTCVPEPASQHTLRSGPGCLQQPEQQGVRDPGGIWAKLGAAEASAERLQGRRSRGASGSE
PQQMGSDVRDLNALLPAVPSLGGGGGCALPVSGAAQWAPVLDFAPPGASAYGSLGGPAPPPAPPPPPPPP
PHSFIKQEPSWGGAEPHEEQCLSAFTVHFSGQFTGTAGACRYGPFGPPPPSQASSGQARMFPNAPYLPSC
LESQPAIRNQGYSTVIFDGTPSYGHTPSHHAAQFPNHSFKHEDPMGQQGSLGEQQYSVPPPVYGCHTPTD
SCTGSQALLLRTPYSSDNLYQMTSQLECMTWNQMNLGATLKGVAAGSSSSVKWIEGQSNHSTGYESDNHT
TPILCGAQYRIHTHGVFRGIQDVRRVPGVAPTLVRSASETSEKRPFMCAYPGQNKRYFKLSHLQMHSRKH
TGEKPYQCDEKDCERRFSRSDQLKRHQRRHTGVKPFQCKTCQRKFSRSDHLKTHTRTHTGKISEKPFSCR
WPSCQKKFARSDELVRHHNMHQRNMTKLQLALMGAPTLPPAWQPFLKDHRISTFKNWPFLEGCACTPERM
AEAGFIHCPTENEPDLAQCFFCFKELEGWEPDDDPIEEHKKHSSGCAFLSVKKQFEELILGEFLKLDRER
AKNKIAKEINNKKKEFEETAEKVRRAIEQLAAMDMQAEGRGTGGSTGDADGPGGPGIPDGPGGNAGGPGE
AGATGGRGPRGAGAARASGPGGGAPRGPHGGAASGLNGCCRCGARGPESRLLEFYLAMPFATPMEAELAR
RSLAQDAPPLPVPGVLLKEFTVSGNILTIRLTAADHRQLQLSISSCLQQLSLLMWITQCFLPVFLAQPPS
GQRRMPLEQRSQHCKPEEGLEARGEALGLVGAQAPATEEQEAASSSSTLVEVTLGEVPAAESPDPPQSPQ
GASSLPTTMNYPLWSQSYEDSSNQEEEGPSTFPDLESEFQAALSRKVAELVHFLLLKYRAREPVTKAEML
GSVVGNWQYFFPVIFSKASSSLQLVFGIELMEVDPIGHLYIFATCLGLSYDGLLGDNQIMPKAGLLIIVL
AIIAREGDCAPEEKIWEELSVLEVFEGREDSILGDPKKLLTQHFVQENYLEYRQVPGSDPACYEFLWGPR
ALVETSYVKVLHHMVKISGGPHISYPPLHEWVLREGEEMERRRLWGSIQSRYISMSVWTSPRRIVELAGQ
SLIKDEALAIAALELLPRELFPPLFMAAFDGRHSQTLKAMVQAWPFTCLPLGVLMKGQHLHLETFKAVLD
GLDVLLAQEVRPRRWKLQVLDLRKNSHQDFWTVWSGNRASLYSFPEPEAAQPMTKKRKVDGLSTEAEQPF
IPVEVLVDLFLKEGACDELFSYLIEKVKRKKNVLRLCCKKLKIFAMPMQDIKMILKMVQLDSIEDLEVTC
TWKLPTLAKFSPYLGQMINLRRLLLSHIHASSYISPEKEEQYIAQFTSQFLSLQCLQALYVDSLFFLRGR
LDQLLRHVMNPLETLSITNCRLSEGDVMHLSQSPSVSQLSVLSLSGVMLIDVSPEPLQALLERASATLQD
LVFDECGITDDQLLALLPSLSHCSQLTTLSFYGNSISISALQSLLQHLIGLSNLTHVLYPVPLESYEDIH
GTLHLERLAYLHARLRELLCELGRPSMVWLSANPCPHCGDRIFYDPEPILCPCFMPN
>ARTIFICIAL: FUSION OF Human CD5 (signal peptide only), WT1, BIRC5,
NY-ESO-1, MAGEA3, and PRAME
SEQ ID NO: 8
MPMGSLQPLATLYLLGMLVASCLGLDFLLLQDPASTCVPEPASQHTLRSGPGCLQQPEQQGVRDPGGIWA
KLGAAEASAERLQGRRSRGASGSEPQQMGSDVRDLNALLPAVPSLGGGGGCALPVSGAAQWAPVLDFAPP
GASAYGSLGGPAPPPAPPPPPPPPPHSFIKQEPSWGGAEPHEEQCLSAFTVHFSGQFTGTAGACRYGPFG
PPPPSQASSGQARMFPNAPYLPSCLESQPAIRNQGYSTVTFDGTPSYGHTPSHHAAQFPNHSFKHEDPMG
QQGSLGEQQYSVPPPVYGCHTPTDSCTGSQALLLRTPYSSDNLYQMTSQLECMTWNQMNLGATLKGVAAG
SSSSVKWTEGQSNHSTGYESDNHTTPILCGAQYRIHTHGVERGIQDVRRVPGVAPTLVRSASETSEKRPE
MCAYPGQNKRYFKLSHLQMHSRKHTGEKPYQCDFKDCERRFSRSDQLKRHQRRHTGVKPFQCKTCQRKES
RSDHLKTHTRTHTGKISEKPFSCRWPSCQKKFARSDELVRHHNMHQRNMTKLQLALMGAPTLPPAWQPFL
KDHRISTFKNWPFLEGCACTPERMAEAGFIHCPTENEPDLAQCFFCFKELEGWEPDDDPIEEHKKASSGC
AFLSVKKQFEELTLGEFLKLDRERAKNKIAKEINNKKKEFEETAEKVRRAIEQLAAMDMQAEGRGTGGST
GDADGPGGPGIPDGPGGNAGGPGEAGATGGRGPRGAGAARASGPGGGAPRGPHGGAASGINGCCRCGARG
PESRLLEFYLAMPFATPMEAELARRSLAQDAPPLPVPGVLLKEFTVSGNILTIRLTAADHRQLQLSISSC
LQQLSLLMWITQCFLPVFLAQPPSGQRRMPLEQRSQHCKPEEGLEARGEALGLVGAQAPATEEQEAASSS
STLVEVTLGEVPAAESPDPPQSPQGASSLPTTMNYPLWSQSYEDSSNQEEEGPSTFPDLESEFQAALSRK
VAELVHFLLLKYRAREPVTKAEMLGSVVGNWQYFFPVIFSKASSSLQLVFGIELMEVDPIGHLYIFATCL
GLSYDGLLGDNQIMPKAGLLIIVLAIIAREGDCAPEEKIWEELSVLEVFEGREDSILGDPKKLLTQHFVQ
ENYLEYRQVPGSDPACYEFLWGPRALVETSYVKVLHHMVKISGGPHISYPPLHEWVLREGEEMERRRLWG
SIQSRYISMSVWTSPRRLVELAGQSLLKDEALAIAALELLPRELFPPLEMAAFDGRHSQTLKAMVQAWPE
TCLPLGVLMKGQHLHLETFKAVLDGLDVLLAQEVRPRRWKLQVLDLRKNSHQDFWTVWSGNRASLYSFPE
PEAAQPMTKKRKVDGLSTEAEQPFIPVEVLVDLFLKEGACDELFSYLIEKVKRKKNVLRLCCKKLKIFAM
PMQDIKMILKMVQLDSIEDLEVTCTWKLPTLAKFSPYLGQMINLRRLLLSHIHASSYISPEKEEQYIAQF
TSQFLSLQCLQALYVDSLFFLRGRLDQLLRHVMNPLETLSITNCRLSEGDVMHLSQSPSVSQLSVLSLSG
VMLTDVSPEPLQALLERASATLQDLVEDECGITDDQLLALLPSLSECSQLTTLSFYGNSISISALQSLLQ
HLIGLSNLTHVLYPVPLESYEDIHGTLHLERLAYLHARLRELLCELGRPSMVWLSANPCPHCGDRTFYDP
EPILCPCEMPN
>ARTIFICIAL: c-srRNA1 nsP2
SEQ ID NO: 9
GSVETPRGLIKVISYDGEDKIGSYAVLSPQAVLKSEKLSCIHPLAEQVIVITHSGRKGRY
AVEPYHGKVVVPEGHAIPVQDFQALSESATIVYNEREFVNRYLHHIATHGGALNTDEEYY
KTVKPSEHDGEYLYDIDRKQCVKKELVTGLGLIGELVDPPFHEFAYESLRTRPAAPYQVP
TIGVYGVPGSGKSGIIKSAVTKKDLVVSAKKENCAEIIRDVKKMKGLDVNARTVDSVLLN
GCKHPVETLYIDEAFACHAGTLRALIAIIRPKKAVLCGDPKQCGFFNMMCLKVHFNHEIC
TQVFHKSISRRCTKSVTSVVSTLFYDKKMRTINPKETKIVIDTTGSTKPKQDDLILTCFR
GWVKQLQIDYKGNEIMTAAASQGLTRKGVYAVRYKVNENPLYAPTSEHVNVLLIRTEDRI
VWKTLAGDPWIKILTAKYPGNFTATIEEWQAEHDAIMRHILERPDPTDVFQNKANVCWAK
ALVPVLKTAGIDMTTEQWNTVDYFETDKAHSAEIVLNQLCVRFFGLDLDSGLFSAPTVPL
SIRNNHWDNSPSPNMYGLNKEVVRQLSRRYPQLPRAVATGRVYDMNTGRAATGTLRNYDP
RINLVPVNRRLPHALVLHHNEHPQSDFSSFVSKLKGRTVLVVGEKLSVPGKMVDWLSDRP
EATFRARLDLGIPGDVPKYDIIFVNVRTPYKYHHYQQCEDHAIKLSMLIKKACLHLNPGG
TCVSIGYGYADRASESIIGAIARQFKFSRVCKPKSSLEETEVLFVFIGYDRKARTHNSYK
LSSTLINIYTGSRLHEAGC
>ARTIFICIAL: c-srRNA3 nsP2
SEQ ID NO: 10
GSVETPRGLIKVTSYAGEDKIGSYAVLSPQAVLKSEKLSCIHPLAEQVIVITHSGRKGRY
AVEPYHGKVVVPEGHAIPVQDFQALSESATIVYNEREFVNRYLHHIATHGGALNTDEEYY
KTVKPSEHDGEYLYDIDRKQCVKKELVTGLGLTGELVDPPFHEFAYESLRTRPAAPYQVP
TIGVYGVPGSGKSGIIKSAVTKKDLVVSAKKENCAEIIRDVKKMKGLDVNARTVDSVLIN
GCKHPVETLYIDEAFACHAGTLRALIAIIRPKKAVLCGDPKQCGFFNMMCLKVHFNHEIC
TQVFHKSISRRCTKSVTSVVSTLFYDKKMRTTNPKETKIVIDTTGSTKPKQDDLILTCFR
GWVKQLQIDYKGNEIMTAAASQGLTRKGVYAVRYKVNENPLYAPTSEHVNVLLTRTEDRI
VWKTLAGDPWIKILTAKYPGNFTATIEEWQAEHDAIMRHILERPDPTDVFQNKANVCWAK
ALVPVLKTAGIDMTTEQWNTVDYFETDKAHSAEIVLNQLCVRFFGLDLDSGLFSAPTVPL
SIRNNHWDNSPSPNMYGINKEVVRQLSRRYPQLPRAVATGRVYDMNTGAAATGTLRNYDP
RINLVPVNRRLPHALVLHHNEHPQSDFSSFVSKLKGRTVLVVGEKLSVPGKMVDWLSDRP
EATFRARLDLGIPGDVPKYDIIFVNVRTPYKYHHYQQCEDHAIKLSMLTKKACLHLNPGG
TCVSIGYGYADRASESIIGAIARQFKFSRVCKPKSSLEETEVLFVFIGYDRKARTHNPYK
LSSTLTNIYTGSRLHEAGC
>ARTIFICIAL: c-srRNA4 nsP2
SEQ ID NO: 11
GSVETPRGLIKVTSYAGEDKIGSYAVLSPQAVLKSEKLSCIHPLAEQVIVITHSGRKGRY
AVEPYHGKVVVPEGHAIPVQDFQALSESATIVYNEREFVNRYLHHIATHGGALNTDEEYY
KTVKPSEHDGEYLYDIDRKQCVKKELVIGLGLIGELVDPPFHEFAYESLRTRPAAPYQVP
TIGVYGVPGSGKSGIIKSAVTKKDLVVSAKKENCAEIIRDVKKMKGLDVNARTVDSVLLN
GCKHPVETLYIDEAFACHAGTLRALIAIIRPKKAVLCGDPKQCGFFNMMCLKVHFNHEIC
TQVFHKSISRRCTKSVTSVVSTLFYDKRMRTINPKETKIEIDTTGSTKPKQDDLILTCER
GWVKQLQIDYKGNEIMTAAASQGLTRKGVYAVRYKVNENPLYAPTSEHVNVLLTRTEDRI
VWKTLAGDPWIKTLTAKYPGNFTATIEEWQAEHDAIMRHILERPDPTDVFQNKANVCWAK
ALVPVLKTAGIDMTTEQWNTVDYFETDKAHSAEIVLNQLCVRFFGLDLDSGLFSAPTVPL
SIRNNHWDNSPSPNMYGLNKEVVRQLSRRYPQLPRAVATGRVYDMNTGAAATGTLRNYDP
RINLVPVNRRLPHALVLHHNEHPQSDFSSFVSKLKGRTVLVVGEKLSVPGKKVDWLSDQP
EATFRARLDIGIPGDVPKYDIVFINVRTPYKYHHYQQCEDHAIKLSMLTKKACLHLNPGG
TCVSIGYGYADRASESIIGAIARQFKFSRVCKPKSSHEETEVLFVFIGYDRKARTHNPYK
LSSTLTNIYTGSRLHEAGC
>ARTIFICIAL: c-srRNA nsP2 consensus
SEQ ID NO: 12
GSVETPRGLIKVTSY[A/D]GEDKIGSYAVLSPQAVLKSEKLSCIHPLAEQVIVITHSGRKGRY
AVEPYHGKVVVPEGHAIPVQDFQALSESATIVYNEREFVNRYLHHIATHGGALNTDEEYY
KTVKPSEHDGEYLYDIDRKQCVKKELVTGLGLTGELVDPPFHEFAYESLRTRPAAPYQVP
TIGVYGVPGSGKSGIIKSAVIKKDLVVSAKKENCAEIIRDVKKMKGLDVNARTVDSVLLN
GCKHPVETLYIDEAFACHAGTLRALIAIIRPKKAVLCGDPKQCGFENMMCLKVHFNHEIC
TQVFHKSISRRCTKSVTSVVSTLFYDK[K/R]MRITNPKETKI[V/E]IDTTGSTKPKQDDLILTCER
GWVKQLQIDYKGNEIMTAAASQGLTRKGVYAVRYKVNENPLYAPTSEHVNVLLTRTEDRI
VWKTLAGDPWIKTILTAKYPGNFTATIEEWQAEHDAIMRHILERPDPTDVFQNKANVCWAK
ALVPVLKTAGIDMTTEQWNTVDYFETDKAHSAEIVLNQLCVRFFGLDLDSGLFSAPTVPL
SIRNNHWDNSPSPNMYGLNKEVVRQLSRRYPQLPRAVATGRVYDMNTGAAATGTLRNYDP
RINLVPVNRRLPHALVLHHNEHPQSDFSSFVSKLKGRTVLVVGEKLSVPGK[M/K]VDWLSD[R/Q]P
EATFRARLDLGIPGDVPKYDI[I/V]F[V/I]NVRTPYKYHHYQQCEDHAIKLSMLTKKACLHLNPGG
TCVSIGYGYADRASESIIGAIARQFKFSRVCKPKSS[L/H]EETEVLFVFIGYDRKARTHN[P/S]YK
LSSTLINIYTGSRLHEAGC
>VEEV: srRNA0
SEQ ID NO: 13
GSVETPRGLIKVTSYAGEDKIGSYAVLSPQAVLKSEKLSCIHPLAEQVIVITHSGRKGRYAVEPYHGKVV
VPEGHAIPVQDFQALSESATIVYNEREFVNRYLHHIATHGGALNTDEEYYKTVKPSEHDGEYLYDIDRKQ
CVKKELVTGLGLTGELVDPPFHEFAYESLRTRPAAPYQVPTIGVYGVPGSGKSGIIKSAVTKKDLVVSAK
KENCAEIIRDVKKMKGLDVNARTVDSVLLNGCKHPVETLYIDEAFACHAGTLRALIAIIRPKKAVLCGDP
KQCGFFNMMCLKVHFNHEICTQVFHKSISRRCTKSVTSVVSTLFYDKKMRTINPKETKIVIDTTGSTKPK
QDDLILTCFRGWVKQLQIDYKGNEIMTAAASQGLIRKGVYAVRYKVNENPLYAPTSEHVNVLLTRTEDRI
VWKTLAGDPWIKTLTAKYPGNFTATIEEWQAEHDAIMRHILERPDPTDVFQNKANVCWAKALVPVLKTAG
IDMTTEQWNTVDYFEIDKAHSAEIVLNQLCVRFFGLDLDSGLFSAPTVPLSIRNNHWDNSPSPNMYGINK
EVVRQLSRRYPQLPRAVATGRVYDMNTGTLRNYDPRINLVPVNRRLPHALVLHHNEHPQSDESSFVSKLK
GRTVLVVGEKLSVPGKMVDWLSDRPEATFRARLDLGIPGDVPKYDIIFVNVRTPYKYHHYQQCEDHAIKL
SMLTKKACLHLNPGGTCVSIGYGYADRASESIIGAIARQFKFSRVCKPKSSLEETEVLFVFIGYDRKART
HNPYKLSSTLINIYTGSRLHEAGC
>ARTIFICAL PROTEIN: TS INSERTION
SEQ ID NO: 14
TGAAA
>ARTIFICIAL (TAA-5107 WITHOUT CD5 SIGNAL PEPTIDE)
SEQ ID NO: 15
MQAEGRGIGGSTGDADGPGGPGIPDGPGGNAGGPGEAGATGGRGPRGAGAARASGPGGGAPRGPHGGAAS
GLNGCCRCGARGPESRLLEFYLAMPFATPMEAELARRSLAQDAPPLPVPGVLLKEFTVSGNILTIRLTAA
DHRQLQLSISSCLQQLSLLMWITQCFLPVFLAQPPSGQRRMPLEQRSQHCKPEEGLEARGEALGLVGAQA
PATEEQEAASSSSTLVEVTLGEVPAAESPDPPQSPQGASSLPITMNYPLWSQSYEDSSNQEEEGPSTFPD
LESEFQAALSRKVAELVHFLLLKYRAREPVTKAEMLGSVVGNWQYFFPVIFSKASSSLQLVFGIELMEVD
PIGHLYIFATCLGLSYDGLLGDNQIMPKAGLLIIVLAIIAREGDCAPEEKIWEELSVLEVFEGREDSILG
DPKKLLTQHFVQENYLEYRQVPGSDPACYEFLWGPRALVETSYVKVLHHMVKISGGPHISYPPLHEWVLR
EGEEMLLAVLYCLLWSFQTSAGHFPRACVSSKNLMEKECCPPWSGDRSPCGQLSGRGSCQNILLSNAPLG
PQFPFTGVDDRESWPSVFYNRTCQCSGNFMGENCGNCKFGFWGPNCTERRLLVRRNIFDLSAPEKDKFFA
YLTLAKHTISSDYVIPIGTYGQMKNGSTPMENDINIYDLFVWMHYYVSMDALLGGSEIWRDIDFAHEAPA
FLPWHRLFLLRWEQEIQKLIGDENFTIPYWDWRDAEKCDICTDEYMGGQHPINPNLLSPASFFSSWQIVC
SRLEEYNSHQSLCNGTPEGPLRRNPGNHDKSRTPRLPSSADVEFCLSLTQYESGSMDKAANFSFRNTLEG
FASPLTGIADASQSSMHNALHIYMNGIMSQVQGSANDPIFLLHHAFVDSIFEQWLRRHRPLQEVYPEANA
PIGHNRESYMVPFIPLYRNGDFFISSKDLGYDYSYLQDSDPDSFQDYIKSYLEQASRIWSWLLGAAMVGA
VLTALLAGLVSLLCRHKRKQLPEEKQPLLMEKEDYHSLYQSHLMNESPDPTDLAGVIIELGPNDSPQTSE
FKGATEEAPAKESPHISEFKGAARVSPISESVLARLSKFEVEDAENVASYDSKIKKIVHSIVSSFAFGLE
GVFLVLLDVTLILADLIFTDSKLYIPLEYRSISLAIALFFLMDVLLRVFVERRQQYFSDLENILDTAIIV
ILLLVDVVYIFFDIKLLRNIPRWTHLLRLLRLIILLRIFHLFHQKRQLEKLIRRRVSENKRRYTRDGFDL
DLTYVTERIIAMSFPSSGRQSFYRNPIKEVVRFLDKKHRNHYRVYNLCSERAYDPKHFHNRVVRIMIDDH
NVPTLHQMVVFTKEVNEWMAQDLENIVAIHCKGGIDRIGTMVCAFLIASEICSTAKESLYYFGERRTDKT
HSEKFQGVKTPSQKRYVAYFAQVKHLYNWNLPPRRILFIKHFIIYSIPRYVRDLKIQIEMEKKVVFSTIS
LGKCSVIDNITTDKILIDVFDGLPLYDDVKVQFFYSNLPTYYDNCSFYFWLHTSFIENNRLYLPKNELDN
LHKQKARRIYPSDFAVEILFGEKMTSSDVVAGSD
>ARTIFICIAL (TAA-5107 WITH CD5 SIGNAL PEPTIDE)
SEQ ID NO: 16
MPMGSLQPLATLYLLGMLVASCLGMQAEGRGIGGSTGDADGPGGPGIPDGPGGNAGGPGEAGATGGRGPR
GAGAARASGPGGGAPRGPHGGAASGINGCCRCGARGPESRLLEFYLAMPFATPMEAELARRSLAQDAPPL
PVPGVLLKEFTVSGNILTIRLTAADHRQLQLSISSCLQQLSLLMWITQCFLPVFLAQPPSGQRRMPLEQR
SQHCKPEEGLEARGEALGLVGAQAPATEEQEAASSSSTLVEVTLGEVPAAESPDPPQSPQGASSLPTTMN
YPLWSQSYEDSSNQEEEGPSTFPDLESEFQAALSRKVAELVHFLLLKYRAREPVTKAEMLGSVVGNWQYE
FPVIFSKASSSLQLVEGIELMEVDPIGHLYIFATCLGLSYDGLLGDNQIMPKAGLLIIVLAIIAREGDCA
PEEKIWEELSVLEVFEGREDSILGDPKKLLTQHEVQENYLEYRQVPGSDPACYEFLWGPRALVETSYVKV
LHHMVKISGGPHISYPPLHEWVLREGEEMLLAVLYCLLWSFQTSAGHFPRACVSSKNLMEKECCPPWSGD
RSPCGQLSGRGSCQNILLSNAPLGPQFPFTGVDDRESWPSVFYNRICQCSGNFMGFNCGNCKFGFWGPNC
TERRLLVRRNIFDLSAPEKDKFFAYLILAKHTISSDYVIPIGTYGQMKNGSTPMENDINIYDLFVWMHYY
VSMDALLGGSEIWRDIDFAHEAPAFLPWHRLFLLRWEQEIQKLTGDENFTIPYWDWRDAEKCDICTDEYM
GGQHPTNPNLLSPASFFSSWQIVCSRLEEYNSHQSLCNGTPEGPLRRNPGNHDKSRTPRLPSSADVEFCL
SLTQYESGSMDKAANFSERNTLEGFASPLTGIADASQSSMHNALHIYMNGTMSQVQGSANDPIFLLHHAF
VDSIFEQWLRRHRPLQEVYPEANAPIGHNRESYMVPFIPLYRNGDFFISSKDLGYDYSYLQDSDPDSFQD
YIKSYLEQASRIWSWLLGAAMVGAVLTALLAGLVSLLCRHKRKQLPEEKQPLLMEKEDYHSLYQSHLMNE
SPDPIDLAGVIIELGPNDSPQTSEFKGATEEAPAKESPHTSEFKGAARVSPISESVLARLSKFEVEDAEN
VASYDSKIKKIVHSIVSSFAFGLFGVFLVLLDVTLILADLIFIDSKLYIPLEYRSISLAIALFFLMDVLL
RVFVERRQQYFSDLFNILDTAIIVILLLVDVVYIFFDIKLLRNIPRWTHLLRLLRLIILLRIFHLFHQKR
QLEKLIRRRVSENKRRYTRDGFDLDLTYVTERIIAMSFPSSGRQSFYRNPIKEVVRFLDKKHRNHYRVYN
LCSERAYDPKHFHNRVVRIMIDDHNVPTLHQMVVFTKEVNEWMAQDLENIVAIHCKGGIDRIGTMVCAFL
IASEICSTAKESLYYFGERRTDKTHSEKFQGVKTPSQKRYVAYFAQVKHLYNWNLPPRRILFIKHFIIYS
IPRYVRDLKIQIEMEKKVVFSTISLGKCSVLDNITTDKILIDVFDGLPLYDDVKVQFFYSNLPTYYDNCS
FYFWLHISFIENNRLYLPKNELDNLHKQKARRIYPSDFAVEILFGEKMTSSDVVAGSD
>ARTIFICIAL (TSA-5109 WITHOUT CD5 SIGNAL PEPTIDE)
SEQ ID NO: 17
YKLVVVGADGVGKSALTYKLVVVGAVGVGKSALTYKLVVVGARGVGKSALTYKLVVVGACGVGKSALTYK
LVVVGAAGVGKSALTYKLVVVGASGVGKSALTKLVVVGAGDVGKSALTIKLVVVGAGCVGKSALTIKLVV
VGAGPVGKSALTIKLVVVGAGSVGKSALTILDILDTAGHEEYSAMRDLDILDTAGKEEYSAMRDLDILDT
AGREEYSAMRD
>ARTIFICIAL (TSA-5109 WITH CD5 SIGNAL PEPTIDE) 245AA
SEQ ID NO: 18
MPMGSLQPLATLYLLGMLVASCLGYKLVVVGADGVGKSALTYKLVVVGAVGVGKSALTYKLVVVGARGVG
KSALTYKLVVVGACGVGKSALTYKLVVVGAAGVGKSALTYKLVVVGASGVGKSALTKLVVVGAGDVGKSA
LTIKLVVVGAGCVGKSALTIKLVVVGAGPVGKSALTIKLVVVGAGSVGKSALTILDILDTAGHEEYSAMR
DLDILDTAGKEEYSAMRDLDILDTAGREEYSAMRD
>ARTIFICIAL (TSA-5111 WITHOUT CD5 SIGNAL PEPTIDE)
SEQ ID NO: 19
MTEYKLVVVGADGVGKSALTIQLIQNMTEYKLVVVGAVGVGKSALTIQLIQNMTEYKLVVVGARGVGKSA
LTIQLIQNMTEYKLVVVGACGVGKSALTIQLIQNMTEYKLVVVGAAGVGKSALTIQLIQNMTEYKLVVVG
ASGVGKSALTIQLIQNMTEYKLVVVGAGDVGKSALTIQLIQNHMTEYKLVVVGAGCVGKSALTIQLIQNH
MTEYKLVVVGAGPVGKSALTIQLIQNHMTEYKLVVVGAGSVGKSALTIQLIQNHDGETCLLDILDTAGHE
EYSAMRDQYMRTGDGETCLLDILDTAGKEEYSAMRDQYMRTGDGETCLLDILDTAGREEYSAMRDQYMRT
G
>ARTIFICIAL (TSA-5111 WITH CD5 SIGNAL PEPTIDE) 375AA
SEQ ID NO: 20
MPMGSLQPLATLYLLGMLVASCLGMTEYKLVVVGADGVGKSALTIQLIQNMTEYKLVVVGAVGVGKSALT
IQLIQNMTEYKLVVVGARGVGKSALTIQLIQNMTEYKLVVVGACGVGKSALTIQLIQNMTEYKLVVVGAA
GVGKSALTIQLIQNMIEYKLVVVGASGVGKSALTIQLIQNMTEYKLVVVGAGDVGKSALTIQLIQNHMTE
YKLVVVGAGCVGKSALTIQLIQNHMTEYKLVVVGAGPVGKSALTIQLIQNHMTEYKLVVVGAGSVGKSAL
TIQLIQNHDGETCLLDILDTAGHEEYSAMRDQYMRTGDGETCLLDILDTAGKEEYSAMRDQYMRIGDGET
CLLDILDTAGREEYSAMRDQYMRTG
>WT-KRAS-32AA
SEQ ID NO: 21
MTEYKLVVVGAGGVGKSALTIQLIQNHFVDEY
>G12D-32AA
SEQ ID NO: 22
MTEYKLVVVGADGVGKSALTIQLIQNHFVDEY
>G12D-17AA
SEQ ID NO: 23
YKLVVVGADGVGKSALT
>G12V-17AA
SEQ ID NO: 24
YKLVVVGAVGVGKSALT
>G12R-17AA
SEQ ID NO: 25
YKLVVVGARGVGKSALT
>G12C-17AA
SEQ ID NO: 26
YKLVVVGACGVGKSALT
>G12A-17AA
SEQ ID NO: 27
YKLVVVGAAGVGKSALT
>G12S-17AA
SEQ ID NO: 28
YKLVVVGASGVGKSALT
>ARTIFICIAL-G12X-POLYPROTEIN-102AA
SEQ ID NO: 29
YKLVVVGADGVGKSALTYKLVVVGAVGVGKSALTYKLVVVGARGVGKSALTYKLVVVGACGVGKSALTYK
LVVVGAAGVGKSALTYKLVVVGASGVGKSALT
>WT-KRAS-39AA
SEQ ID NO: 30
KQVVIDGETCLLDILDTAGQEEYSAMRDQYMRTGEGFLC
>Q61H-KRAS-39AA
SEQ ID NO: 31
KQVVIDGETCLLDILDTAGHEEYSAMRDQYMRIGEGFLC
>Q61H-KRAS-29AA
SEQ ID NO: 32
DGETCLLDILDTAGHEEYSAMRDQYMRTG
>Q61K-KRAS-29AA
SEQ ID NO: 33
DGETCLLDILDTAGKEEYSAMRDQYMRTG
>Q61R-KRAS-29AA
SEQ ID NO: 34
DGETCLLDILDTAGREEYSAMRDQYMRTG
>ARTIFICIAL-Q61X-POLYPROTEIN-87AA
SEQ ID NO: 35
DGETCLLDILDTAGHEEYSAMRDQYMRTGDGETCLLDILDTAGKEEYSAMRDQYMRTGDGETCLLDILDT
AGREEYSAMRDQYMRIG
>G13D-KRAS-17AA
SEQ ID NO: 36
KLVVVGAGDVGKSALTI
>G13C-KRAS-17AA
SEQ ID NO: 37
KLVVVGAGCVGKSALTI
>G13P-KRAS-17AA
SEQ ID NO: 38
KLVVVGAGPVGKSALTI
>G13S-KRAS-17AA
SEQ ID NO: 39
KLVVVGAGSVGKSALTI
>Q61H-KRAS-17AA
SEQ ID NO: 40
LDILDTAGHEEYSAMRD
>Q61K-KRAS-17AA
SEQ ID NO: 41
LDILDTAGKEEYSAMRD
>Q61R-KRAS-17AA
SEQ ID NO: 42
LDILDTAGREEYSAMRD
>G12D-KRAS-26AA
SEQ ID NO: 43
MTEYKLVVVGADGVGKSALTIQLIQN
>G12V-KRAS-26AA
SEQ ID NO: 44
MTEYKLVVVGAVGVGKSALTIQLIQN
>G12R-KRAS-26AA
SEQ ID NO: 45
MTEYKLVVVGARGVGKSALTIQLIQN
>G12C-KRAS-26AA
SEQ ID NO: 46
MTEYKLVVVGACGVGKSALTIQLIQN
>G12A-KRAS-26AA
SEQ ID NO: 47
MTEYKLVVVGAAGVGKSALTIQLIQN
>G12S-KRAS-26AA
SEQ ID NO: 48
MTEYKLVVVGASGVGKSALTIQLIQN
>G13D-KRAS-27AA
SEQ ID NO: 49
MTEYKLVVVGAGDVGKSALTIQLIQNH
>G13C-KRAS-27AA
SEQ ID NO: 50
MTEYKLVVVGAGCVGKSALTIQLIQNH
>G13P-KRAS-27AA
SEQ ID NO: 51
MTEYKLVVVGAGPVGKSALTIQLIQNH
>KRAS-G13S-27AA
SEQ ID NO: 52
MTEYKLVVVGAGSVGKSALTIQLIQNH
>ARTIFICIAL
SEQ ID NO: 53
MTEYKLVVVGAX₁GVGKSALTIQLIQNXaXbXcXdXeXf
MTEYKLVVVGAX₂GVGKSALTIQLIQNXaXbXcXdXeXf
MTEYKLVVVGAX₃GVGKSALTIQLIQNXaXbXcXdXeXf
MTEYKLVVVGAX₄GVGKSALTIQLIQNXaXbXcXdXexf
MTEYKLVVVGAX₅GVGKSALTIQLIQNXaXbXcXdXeXf
MTEYKLVVVGAX₆GVGKSALTIQLIQNXaXbXcXdXeXf,
wherein X₁, X₂, X₃, X₄, X₅, and X₆ are independently selected from D,
V, R, C, A, and S, and wherein Xa, Xb, Xc, Xd, Xe, and Xf are
independently selected from G, S, and absent.
>ARTIFICIAL
SEQ ID NO: 54
MTEYKLVVVGAGX₁₀VGKSALTIQLIQNHXaXbXcXdXeXf
MTEYKLVVVGAGX₁₁VGKSALTIQLIQNHXaXbXcXdXeXf
MTEYKLVVVGAGX₁₂VGKSALTIQLIQNHXaXbXcXdXeXf
MTEYKLVVVGAGX₁₃VGKSALTIQLIQNHXaXbXcXdXeXf,
wherein X₁₀, X₁₁, X₁₂, and X₁₃, are independently selected from D, C,
P, and S, and wherein Xa, Xb, Xc, Xd, Xe, and Xf are independently
selected from G, S, and absent.
>ARTIFICIAL
SEQ ID NO: 55
DGETCLLDILDTAGX₇EEYSAMRDQYMRTGXaXbXcXdXeXf
DGETCLLDILDTAGX₈EEYSAMRDQYMRTGXaXbXcXdXeXf
DGETCLLDILDTAGX₉EEYSAMRDQYMRTGXaXbXcXdXeXf,
wherein X₇, X₈, and X₉, are independently selected from H, K, and
R, and wherein Xa, Xb, Xc, Xd, Xe, and XE are independently selected
from G, S, and absent.
ARTIFICIAL
SEQ ID NO: 56
YKLVVVGAX₁GVGKSALTXaXbXcXdXeXfYKLVVVGAX₂GVGKSALTXaXbXcXdXexf
YKLVVVGAX₃GVGKSALTXaXbXcXdXeXfYKLVVVGAX₄GVGKSALTXaXbXcXdXeXf
YKLVVVGAX₅GVGKSALTXaXbXcXdXeXfYKLVVVGAX₆GVGKSALT,
wherein X₁, X₂, X₃, X₄, X₅, and X₆ are independently selected from D,
V, R, C, A, and S, and wherein Xa, Xb, Xc, Xd, Xe, and Xf are
independently selected from G, S, and absent.
>ARTIFICIAL
SEQ ID NO: 57
KLVVVGAGX₁₀VGKSALTIXaXbXcXdXeXfKLVVVGAGX₁₁VGKSALTIXaXbXcXdXeXf
KLVVVGAGX₁₂VGKSALTIXaXbXcXdXeXfKLVVVGAGX₁₃VGKSALTI,
wherein X₁₀, X₁₁, X₁₂, and X₁₃, are independently selected from D, C,
P, and S, and wherein Xa, Xb, Xc, Xd, Xe, and Xf are independently
selected from G, S, and absent.
>ARTIFICIAL
SEQ ID NO: 58
LDILDTAGX₇HEEYSAMRDXaXbXcXdXeXfLDILDTAGX₈HEEYSAMRDXaXbXcXdXeXf
LDILDTAGX₉HEEYSAMRD,
wherein X₇, X₈, and X₉, are independently selected from H, K, and
R, and wherein Xa, Xb, Xc, Xd, Xe, and Xf are independently selected
from G, S, and absent .
>G12G
SEQ ID NO: 59
MTEYKLVVVGAGGVGKSALTIQLIQ
G12D
SEQ ID NO: 60
MTEYKLVVVGADGVGKSALTIQLIQ
G12V
SEQ ID NO: 61
MTEYKLVVVGAVGVGKSALTIQLIQ
G12C
SEQ ID NO: 62
MTEYKLVVVGACGVGKSALTIQLIQ

We claim:

1. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:
a) a first segment comprising:
(SEQ ID NO: 53)
MTEYKLVVVGAX₁GVGKSALTIQLIQNXaXbXcXdXeXf
MTEYKLVVVGAX₂GVGKSALTIQLIQNXaXbXcXdXeXf
MTEYKLVVVGAX₃GVGKSALTIQLIQNXaXbXcXdXeXf
MTEYKLVVVGAX₄GVGKSALTIQLIQNXaXbXcXdXeXf
MTEYKLVVVGAX₅GVGKSALTIQLIQNXaXbXcXdXeXf
MTEYKLVVVGAX₆GVGKSALTIQLIQNXaXbXcXdXeXf;
b) a second segment comprising:
(SEQ ID NO: 53)
MTEYKLVVVGAGX₁₀VGKSALTIQLIQNHXaXbXcXdXeXf
MTEYKLVVVGAGX₁₁VGKSALTIQLIQNHXaXbXcXdXeXf
MTEYKLVVVGAGX₁₂VGKSALTIQLIQNHXaXbXcXdXeXf
MTEYKLVVVGAGX₁₃VGKSALTIQLIQNHXaXbXcXdXeXf;
and
c) a third segment comprising:
(SEQ ID NO: 55)
DGETCLLDILDTAGX₇EEYSAMRDQYMRTGXaXbXcXdXeXf
DGETCLLDILDTAGX₈EEYSAMRDQYMRTGXaXbXcXdXeXf
DGETCLLDILDTAGX₉EEYSAMRDQYMRTGXaXbXcXdXeXf,
wherein the first segment, the second segment and the third segment are arranged in any order,
wherein X₁, X₂, X₃, X₄, X₅, and X₆ are independently selected from D, V, R, C, A, and S,
wherein X₇, X₈, and X₉, are independently selected from H, K, and R,
wherein X₁₀, X₁₁, X₁₂, and X₁₃, are independently selected from D, C, P, and S, and
wherein Xa, Xb, Xc, Xd, Xe, and Xf are independently selected from G, S, and absent.

2. The RNA molecule of claim 1, wherein the amino acid sequence of the KRAS polyprotein comprises SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO: 32, SEQ ID NO:33, and SEQ ID NO:34.

3. The RNA molecule of claim 2, wherein the amino acid sequence of the KRAS polyprotein comprises residues 25-375 of SEQ ID NO:20.

4. An RNA molecule comprising an open reading frame (ORF) encoding a fusion protein. wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:
a) a first segment comprising:
(SEQ ID NO: 56)
YKLVVVGAX₁GVGKSALTXaXbXcXdXeXf
YKLVVVGAX₂GVGKSALTXaXbXcXdXeXf
YKLVVVGAX₃GVGKSALTXaXbXcXdXeXf
YKLVVVGAX₄GVGKSALTXaXbXcXdXeXf
YKLVVVGAX₅GVGKSALTXaXbXcXdXeXf
YKLVVVGAX₆GVGKSALT,
b) a second segment comprising:
(SEQ ID NO: 57)
KLVVVGAGX₁₀VGKSALTIXaXbXcXdXeXf
KLVVVGAGX₁₁VGKSALTIXaXbXcXdXeXf
KLVVVGAGX₁₂VGKSALTIXaXbXcXdXeXf
KLVVVGAGX₁₃VGKSALTI,
and
c) a third segment comprising:
(SEQ ID NO: 58)
LDILDTAGX₇HEEYSAMRDXaXbXcXdXeXf
LDILDTAGX₈HEEYSAMRDXaXbXcXdXeXf
LDILDTAGX₉HEEYSAMRD,
wherein the first segment, the second segment and the third segment are arranged in any order,
wherein X₁, X₂, X₃, X₄, X₅, and X₆ are independently selected from D, V, R, C, A, and S,
wherein X₇, X₈, and X₉, are independently selected from H, K, and R,
wherein X₁₀, X₁₁, X₁₂, and X₁₃, are independently selected from D, C, P, and S, and
wherein Xa, Xb, Xc, Xd, Xe, and Xf are independently selected from G, S, and absent.

5. The RNA molecule of claim 4, wherein the amino acid sequence of the KRAS polyprotein comprises SEQ ID NO:23, SEQ ID NO:24, SEQ ID NO:25, SEQ ID NO:26, SEQ ID NO:27, SEQ ID NO:28, SEQ ID NO:36, SEQ ID NO:37, SEQ ID NO:38, SEQ ID NO:39, SEQ ID NO: 40, SEQ ID NO:41, and SEQ ID NO:42.

6. The RNA molecule of claim 5, wherein the amino acid sequence of the KRAS polyprotein comprises residues 25-245 of SEQ ID NO:18.

7. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:
(SEQ ID NO: 56)
YKLVVVGAX₁GVGKSALTXaXbXcXdXeXf
YKLVVVGAX₂GVGKSALTXaXbXcXdXeXf
YKLVVVGAX₃GVGKSALTXaXbXcXdXeXf
YKLVVVGAX₄GVGKSALTXaXbXcXdXeXf
YKLVVVGAX₅GVGKSALTXaXbXcXdXeXf
YKLVVVGAX₆GVGKSALT,
wherein X₁, X₂, X₃, X₄, X₅, and X₆ are independently selected from D, V, R, C, A, and S, and
wherein Xa, Xb, Xc, Xd, Xe, and Xf are independently selected from G, S, and absent.

8. The RNA molecule of claim 7, wherein the amino acid sequence of the KRAS polyprotein comprises SEQ ID NO:23, SEQ ID NO:24, SEQ ID NO:25, SEQ ID NO:26, SEQ ID NO:27, and SEQ ID NO:28.

9. The RNA molecule of claim 8, wherein the amino acid sequence of the KRAS polyprotein comprises SEQ ID NO:29.

10. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:
(SEQ ID NO: 55)
DGETCLLDILDTAGX₇EEYSAMRDQYMRTGXaXbXcXdXeXf
DGETCLLDILDTAGX₈EEYSAMRDQYMRTGXaXbXcXdXeXf
DGETCLLDILDTAGX₉EEYSAMRDQYMRTGXaXbXcXdXeXf,
wherein X₇, X₈, and X₉, are independently selected from H, K, and R, and
wherein Xa, Xb, Xc, Xd, Xe, and Xf are independently selected from G, S, and absent.

11. The RNA molecule of claim 10, wherein the amino acid sequence of the KRAS polyprotein comprises SEQ ID NO:32, SEQ ID NO:33, and SEQ ID NO:34.

12. The RNA molecule of claim 11, wherein the amino acid sequence of the KRAS polyprotein comprises SEQ ID NO:35.

13. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:
(SEQ ID NO: 57)
KLVVVGAGX₁₀VGKSALTIXaXbXcXdXeXf
KLVVVGAGX₁₁VGKSALTIXaXbXcXdXeXf
KLVVVGAGX₁₂VGKSALTIXaXbXcXdXeXf
KLVVVGAGX₁₃VGKSALTI,
wherein X₁₀, X₁₁, X₁₂, and X₁₃, are independently selected from D, C, P, and S, and
wherein Xa, Xb, Xc, Xd, Xe, and Xf are independently selected from G, S, and absent.

14. The RNA molecule of claim 13, wherein the amino acid sequence of the KRAS polyprotein comprises SEQ ID NO:36, SEQ ID NO:37, SEQ ID NO:38, and SEQ ID NO:39.

15. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:
(SEQ ID NO: 58)
LDILDTAGX₇HEEYSAMRDXaXbXcXdXeXf
LDILDTAGX₈HEEYSAMRDXaXbXcXdXeXf
LDILDTAGX₉HEEYSAMRD,
wherein X₇, X₈, and X₉, are independently selected from H, K, and R, and
wherein Xa, Xb, Xc, Xd, Xe, and Xf are independently selected from G, S, and absent.

16. The RNA molecule of claim 15, wherein the amino acid sequence of the KRAS polyprotein comprises SEQ ID NO:40, SEQ ID NO:41, and SEQ ID NO:42.

17. The RNA molecule of claim 8, claim 14, or claim 16, wherein the amino acid sequence of the KRAS polyprotein comprises residues 25-245 of SEQ ID NO:18.

18. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the
(SEQ ID NO: 53)
MTEYKLVVVGAX₁GVGKSALTIQLIQNXaXbXcXdXeXf
MTEYKLVVVGAX₂GVGKSALTIQLIQNXaXbXcXdXeXf
MTEYKLVVVGAX₃GVGKSALTIQLIQNXaXbXcXdXeXf
MTEYKLVVVGAX₄GVGKSALTIQLIQNXaXbXcXdXeXf
MTEYKLVVVGAX₅GVGKSALTIQLIQNXaXbXcXdXeXf
MTEYKLVVVGAX₆GVGKSALTIQLIQNXaXbXcXdXeXf,
wherein X₁, X₂, X₃, X₄, X₅, and X₆ are independently selected from D, V, R, C, A, and S, and
wherein Xa, Xb, Xc, Xd, Xe, and Xf are independently selected from G, S, and absent.

19. The RNA molecule of claim 18, wherein the amino acid sequence of the KRAS polyprotein comprises SEQ ID NO:43, SEQ ID NO:44, SEQ ID NO:45, SEQ ID NO:46, SEQ ID NO: 47, and SEQ ID NO:48.

20. An RNA molecule comprising an open reading frame (ORF) encoding a fusion protein, wherein the ORF comprises from 5′ to 3′:
(i) a nucleotide sequence encoding a mammalian signal peptide; and
(ii) a nucleotide sequence encoding a cancer antigen,
wherein the cancer antigen comprises a KRAS polyprotein and the amino acid sequence of the KRAS polyprotein comprises:
(SEQ ID NO: 54)
MTEYKLVVVGAGX₁₀VGKSALTIQLIQNHXaXbXcXdXeXf
MTEYKLVVVGAGX₁₁VGKSALTIQLIQNHXaXbXcXdXeXf
MTEYKLVVVGAGX₁₂VGKSALTIQLIQNHXaXbXcXdXeXf
MTEYKLVVVGAGX₁₃VGKSALTIQLIQNHXaXbXcXdXeXf,
wherein X₁₀, X₁₁, X₁₂, and X₁₃, are independently selected from D, C, P, and S, and
wherein Xa, Xb, Xc, Xd, Xe, and Xf are independently selected from G, S, and absent.

21. The RNA molecule of claim 20, wherein the amino acid sequence of the KRAS polyprotein comprises SEQ ID NO:49, SEQ ID NO:50, SEQ ID NO:51, and SEQ ID NO:52.

22. The RNA molecule of claim 11, claim 19, or claim 21, wherein the amino acid sequence of the KRAS polyprotein comprises residues 25-375 of SEQ ID NO:20.

23. The RNA molecule of any one of claims 1-22, wherein the mammalian signal peptide is a signal peptide of a surface protein expressed in mammalian antigen presenting cells.

24. The RNA molecule of claim 23, wherein the mammalian signal peptide is a CD5 signal peptide and the amino acid sequence of the CD5 signal peptide comprises SEQ ID NO:1, or the amino acid sequence at least 90% or 95% identical to SEQ ID NO:1.

25. The RNA molecule of any one of claims 1-24, comprising at least one modified nucleoside, optionally wherein the at least one modified nucleoside comprises pseudouridine.

26. A DNA template for the RNA molecule of any one of claims 1-25, optionally wherein a first restriction enzyme site is present upstream of the nucleotide sequence encoding the mammalian signal peptide, and a second restriction site is present downstream of the nucleotide sequence encoding the cancer antigen.

27. An expression vector comprising the DNA template of claim 26.

28. A host cell comprising the expression vector of claim 27.

29. The RNA molecule of any one of claims 1-25, wherein the RNA molecule is a self-replicating RNA.

30. A composition for stimulating an immune response against a cancer antigen in a mammalian subject, comprising an excipient, and the temperature-sensitive self-replicating RNA of claim 29, wherein the self-replicating RNA is a temperature-sensitive RNA that further comprises an Alphavirus replicon lacking a viral structural protein coding region, and wherein the temperature-sensitive self-replicating RNA is capable of expressing the fusion protein at a permissive temperature but not at a non-permissive temperature.

31. A composition for stimulating an immune response against a cancer antigen in a mammalian subject, comprising an excipient, and a temperature-sensitive self-replicating RNA comprising an open reading frame (ORF) encoding a fusion protein, and an Alphavirus replicon lacking a viral structural protein coding region, wherein the ORF comprises from 5′ to 3′:
(i) a nucleotide sequence encoding a mammalian signal peptide; and
(ii) a nucleotide sequence encoding a cancer antigen,
wherein the temperature-sensitive self-replicating RNA is capable of expressing the fusion protein at a permissive temperature but not at a non-permissive temperature, and the cancer antigen comprises a NY-ESO-1 antigen, a MAGEA3 antigen, a TYR antigen, and a TPTE antigen.

32. The composition of claim 31, wherein the mammalian signal peptide is a signal peptide of a surface protein expressed in mammalian antigen presenting cells.

33. The composition of claim 32, wherein the mammalian signal peptide is a CD5 signal peptide and the amino acid sequence of the CDS signal peptide comprises SEQ ID NO: 1, or the amino acid sequence at least 90% or 95% identical to SEQ ID NO:1.

34. The composition of claim 32, wherein the amino acid sequence of the fusion protein comprises SEQ ID NO: 16, or the amino acid sequence at least 90% or 95% identical to SEQ ID NO: 16.

35. The composition of any one of claims 30-34, wherein the Alphavirus is selected from the group consisting of a Venezuelan equine encephalitis virus, a Sindbis virus, and a Semliki Forrest virus.

36. The composition of claim 35, wherein the Alphavirus is a Venezuelan equine encephalitis virus.

37. The composition of any one of claims 30-36, wherein the Alphavirus replicon comprises a nonstructural protein coding region with an insertion of 12-18 nucleotides resulting in expression of a nonstructural Protein 2 (nsP2) comprising from 4 to 6 additional amino acids between beta sheet 5 and beta sheet 6 of the nsP2.

38. The composition of claim 37, wherein the additional amino acids comprise the sequence of SEQ ID NO:14 (TGAAA).

39. The composition of claim 38, wherein the amino acid sequence of the nsP2 comprises SEQ ID NO:12.

40. The composition of claim 39, wherein the amino acid sequence of the nsP2 comprises one sequence selected from the group consisting of SEQ ID NO:9, SEQ ID NO:10, and SEQ ID NO: 11.

41. The composition of claim 40, wherein the amino acid sequence of the nsP2 comprises SEQ ID NO:11.

42. The composition of any one of claims 30-41, wherein the permissive temperature is from 30° C. to 36° C., or 31° C. to 35° C., or 32° C. to 34° C., or 33° C.±0.5° C., and the non-permissive temperature is 37° C.±0.5° C., optionally wherein the permissive temperature is from 31° C. to 35° C. and the non-permissive temperature is at least 37° C.±0.5° C.

43. The composition of any one of claims 30-42, wherein the composition does not comprise lipid nanoparticles.

44. The composition of any one of claims 30-43, wherein the composition further comprises chitosan.

45. A method for stimulating an immune response against a cancer antigen in a mammalian subject, comprising administering the composition of any one of claims 30-44 to a mammalian subject so as to stimulate an immune response against the cancer antigen in the mammalian subject.

46. The method of claim 45, wherein the composition is administered intradermally.

47. The method of claim 45 or claim 46, wherein the immune response comprises a cellular immune response reactive with mammalian cells expressing the cancer antigen.

48. The method of claim 47, wherein the cellular immune response comprises one or both of a cancer antigen-specific cytotoxic T lymphocyte response and a cancer antigen-specific helper T lymphocyte response.

49. The method of claim 48, wherein the immune response further comprises a humoral immune response reactive with the cancer antigen.

50. The method of any one of claims 45-49, wherein the mammalian subject is a human subject.

51. A kit comprising:
(i) the composition of any one of claims 30-44; and
(ii) a device for intradermal delivery of the composition to a mammalian subject.

52. The kit of claim 51, wherein the device comprises a syringe and a needle.

53. A method of expressing a fusion protein, comprising contacting a mammalian cell with the RNA molecule of any one of claims 1-25.

54. The method of claim 53, wherein the contacting is in vitro.

55. The method of claim 53, wherein the contacting is in vivo.

56. A method of treating cancer, comprising administering an effective amount of the composition of any one of claims 30-44 to a mammalian subject in need thereof to treat the cancer.

57. The method of claim 56, wherein cells of the cancer express a KRAS oncogene comprising a substitution at one or more of KRAS positions 12, 13 and 61.

58. The method of claim 56, wherein cells of the cancer express one or more of a NY-ESO-1 antigen, a MAGEA3 antigen, a TYR antigen, and a TPTE antigen.

59. The method of any one of claims 56-58, wherein the composition is administered intradermally.
